(19) United States
(12) Patent Application Publication
(10) Pub. No.: US 2023/0320817 A1
(43) Pub. Date: Oct. 12, 2023

(54) ORTHODONTIC ALIGNMENT SYSTEM AND METHOD

(71) Applicant: Luke Daniel Leviton, Walnut, MS (US)

(72) Inventor: Luke Daniel Leviton, Walnut, MS (US)

(21) Appl. No.: 17/966,768

(22) Filed: Oct. 14, 2022

Related U.S. Application Data
(60) Provisional application No. 63/255,940, filed on Oct. 14, 2021.

(51) Int. Cl. A61C 7/00; A61C 7/08

(52) U.S. Cl. CPC A61C 7/002; A61C 7/08

(57) ABSTRACT

The present invention provides a method of moving at least one tooth within a plurality of teeth. Pursuant to the method, one must determine the mass of the tooth and determine the initial position of the tooth. One then calculates a first force necessary to move the tooth a given distance in a set period of time using Newtonian physics equations, as discussed further below. The calculated force is then applied to the tooth for the given period of time by using an orthodontic aligner tray shaped to apply the calculated force to the tooth. After the expiration of the given period of time, the new position of the tooth is determined, and the Newtonian physics equations are calibrated by adjusting the equations to compensate difference between the actual position of the tooth as compared to the predicted position of the tooth under the previously calculated force. The calibrated equations are then used to calculate a second force needed to move the tooth a second distance in a second period of time, and that force is applied to the tooth using an aligner tray designed to apply the force to the tooth.

### BACKGROUND OF THE INVENTION

### Field of the Invention

The present invention relates to orthodontics and more particularly, but not by way of limitation, to an improved device and method for aligning teeth using alignment trays.

### Discussion of the Prior Art

It is becoming increasingly popular to use a series of molds for orthodontic treatment of teeth. Each mold or tray in the series is used to force teeth to move into their new and improved positions. However, this process suffers from a number of drawbacks. These drawbacks include utilization of unreliable treatment algorithms, failure to properly supervise the patient during movement of teeth, the lack of information available relative to dentition movement, increased cost, orthodontic office directed lab fees, and the need for a patient to physically be present in the orthodontic office on a monthly basis. Prior art methods of analysis become impractical at times, such as when a global pandemic closes all dental/orthodontic offices over the course of months, leaving no way to effectively track and treat a patient. Using prior art methods, sometimes a patient is quoted a total orthodontic treatment time frame, and that time frame runs out with no known reason as to why movement has not been efficacious. Such failures serve to increase the overhead within the orthodontic office and cause unnecessary stress on the patient and practitioner. These limitations of prior art techniques result in the practice of orthodontics being as much art as it is science. The present invention is designed to counter these limitations of the prior art.

### BRIEF DESCRIPTION OF THE DRAWINGS

The accompanying drawings, which are incorporated into and form a part of the specification, illustrate one or more embodiments of the present invention and, together with this description, serve to explain the principles of the invention. The drawings merely illustrate preferred embodiments of the invention and are not to be construed as limiting the scope of the invention.

FIG. 1 is a perspective view of a mouthpiece constructed in accordance with a preferred embodiment of the present invention.

FIG. 2 is a cross-sectional, perspective view of the mouthpiece of FIG. 1.

FIG. 3 is a perspective view of a portion of the mouthpiece of FIG. 1.

FIG. 4 is a plan view of a portion of the mouthpiece of FIG. 1.

FIG. 5 is a side view of a portion of the mouthpiece of FIG. 1.

FIG. 6 is a perspective view of a portion of the mouthpiece of FIG. 1.

FIG. 7 is a plan view of a portion of the mouthpiece of FIG. 1.

FIG. 8 is a side view of a portion of the mouthpiece of FIG. 1.

FIG. 9 is a perspective view of a portion of the mouthpiece of FIG. 1.

FIG. 10 is a side view of a portion of the mouthpiece of FIG. 1.

FIG. 11 is a plan view of a portion of the mouthpiece of FIG. 1.

FIG. 12 is a perspective view of a portion of the mouthpiece of FIG. 1.

FIG. 13 is a side view of a portion of the mouthpiece of FIG. 1.

FIG. 14 is a plan view of a portion of the mouthpiece of FIG. 1.

FIG. 15 is a perspective view of bite registration material.

FIG. 16 is a plan view of bite registration material.

FIG. 17 is a side view of bite registration material.

FIG. 18 is a perspective view of an aligner tray constructed in accordance with a preferred embodiment of the present invention.

FIG. 19 is a plan view of a mouthpiece constructed in accordance with a preferred embodiment of the present invention.

FIG. 20 is a detail taken from FIG. 19 where indicated.

FIG. 21 is schematic for a computing device.

FIG. 22 is a flowchart of a method used in a preferred embodiment of the present invention.

FIG. 23 is a flowchart of a method used in a preferred embodiment of the present invention.

FIG. 24 is a flowchart of a method used in a preferred embodiment of the present invention.

FIG. 25 is a listing of formulas useful for the present invention.

FIG. 26 is a listing of formulas useful for the present invention.

FIG. 27 illustrates the use of triangles and angles to find rotation calculations for a preferred embodiment of the present invention.

FIG. 28 illustrates the use of triangles and angles to find rotation calculations for a preferred embodiment of the present invention.

FIG. 29 illustrates the use of triangles and angles to find rotation calculations for a preferred embodiment of the present invention.

FIGS. 30A, 30B and 30C illustrate the measurements of the present invention in physical context and incorporates the usage of silicone rubber molds to for accurate fitment of the device for multiple arch sizes within the singular arch mouthpiece and or whole mouth mouthpiece.

FIGS. 31A, 31B and 31C illustrate the usage of the speed of sound in reference to the mouth to allocate for usage of ultrasonic, infrasonic and or all forms of mechanical wave.

FIG. 32 displays the adjustment process of the singular arch mouthpiece.

FIGS. 33A, 33B, 33C, 33D, and 33E displays the addition and relation of Newtonian physics equations to calculate and predict tooth movement, rotation, and force application relative to position, displacement, and mass.

FIGS. 34A and 34B illustrates the usage and process of bite registration material to achieve remote monitoring of the full dentition.

FIGS. 35A and 35B illustrates the general circuit layout of the CH101 Echo Ultrasonic sensor array.

FIGS. 36A, 36B, and 36C illustrate the exemplary methods of Binary phase key modulation to achieve optical/mechanical micro distance sensor accuracy.

FIG. 37 illustrates the usage of a pressure membrane to determine pressure distributions in the alignment of the dentition.

FIG. 38A, 38B, 38C, and 38D illustrate the circuit layout/ of the precise optical and mechanical ultrasonic chip-based sensor.

FIG. 39 illustrates the data flow necessary within all forms of analysis.

FIG. 40 illustrates the utilization of positional observation.

FIG. 41 illustrates the CH101 Ultrasonic sensor Chirp terminal.

### DETAILED DESCRIPTION OF THE PREFERRED EMBODIMENTS

Reference is made herein to the drawings. Like reference numerals are used throughout the drawings to depict like or similar elements of the present invention. For the purposes of presenting a brief and clear description of the present invention, multiple embodiments will be discussed as used for orthodontic treatment. These embodiments are exemplary only. Other embodiments and variations will become apparent to those skilled in the art upon study of the specification and drawings of this application.

The present invention provides a method of moving at least one tooth within a plurality of teeth. Pursuant to the method, one must determine the mass of the tooth and determine the initial position of the tooth. One then calculates a first force necessary to move the tooth a given distance in a set period of time using Newtonian physics equations, as discussed further below. The calculated force is then applied to the tooth for the given period of time by using an orthodontic aligner tray shaped to apply the calculated force to the tooth. After the expiration of the given period of time, the new position of the tooth is determined, and the Newtonian physics equations are calibrated by adjusting the equations to compensate difference between the actual position of the tooth as compared to the predicted position of the tooth under the previously calculated force. The calibrated equations are then used to calculate a second force needed to move the tooth a second distance in a second period of time, and that force is applied to the tooth using an aligner tray designed to apply the force to the tooth.

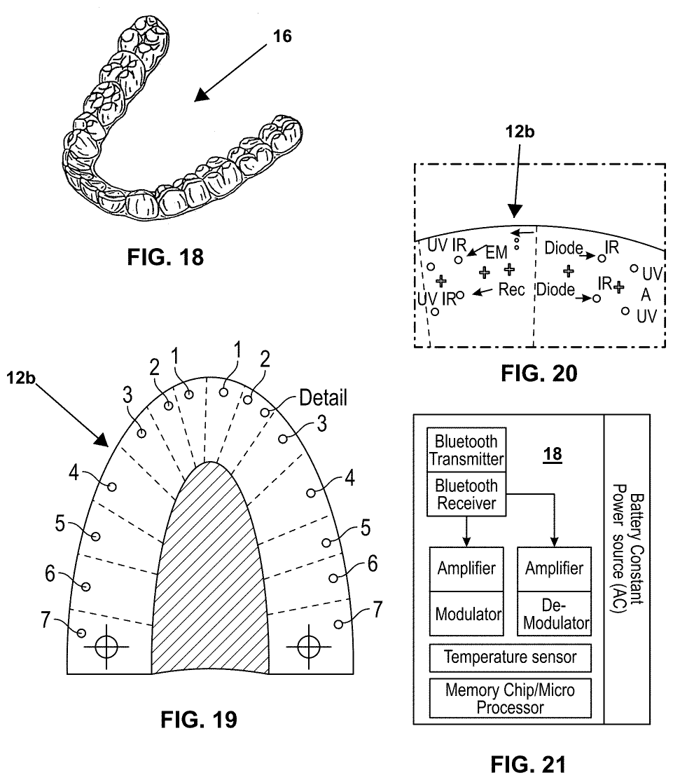

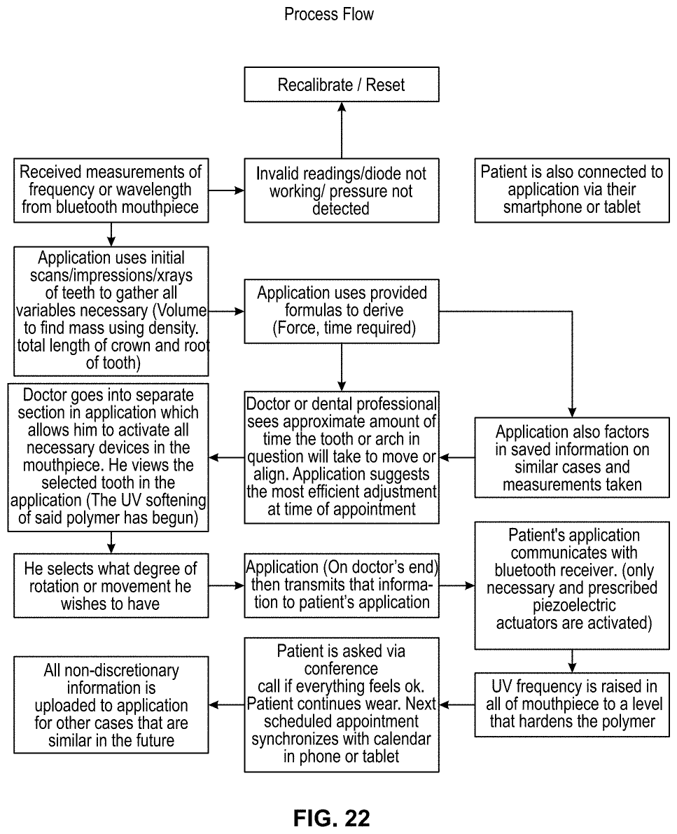

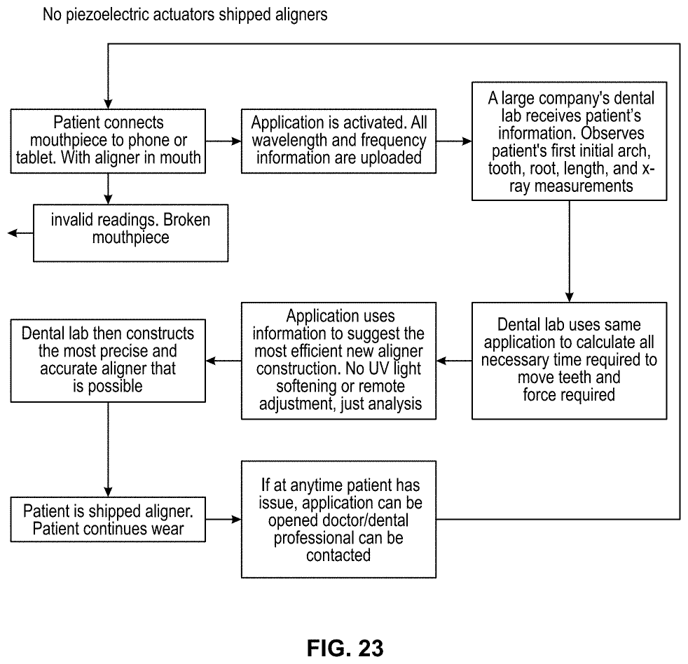

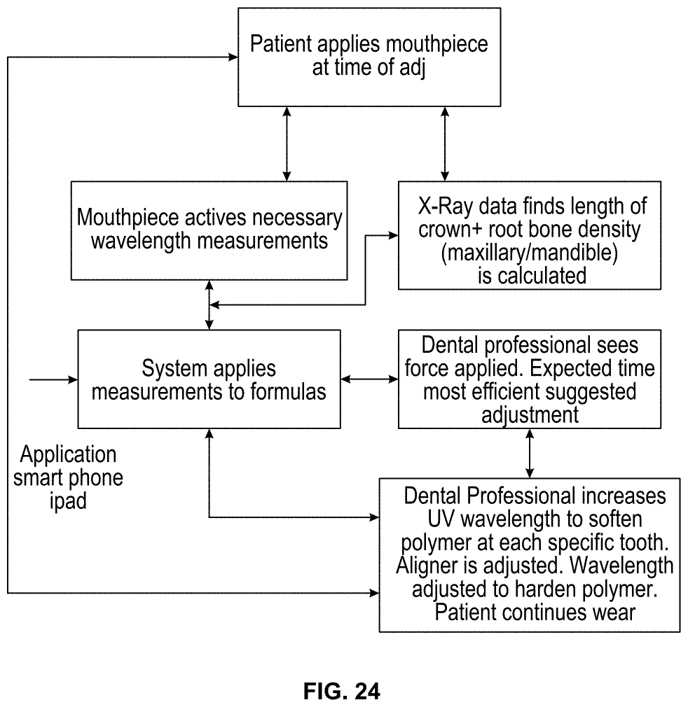

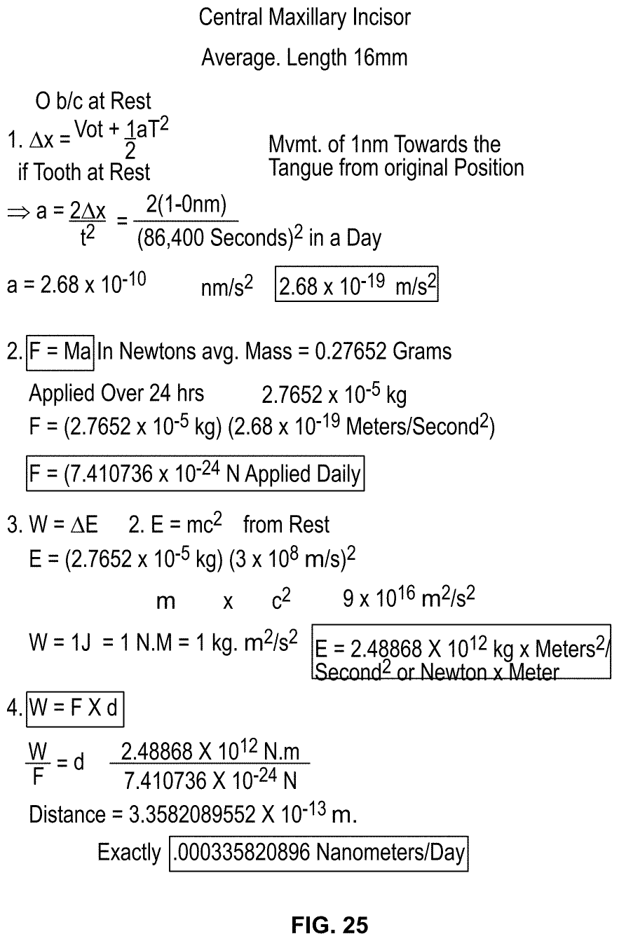

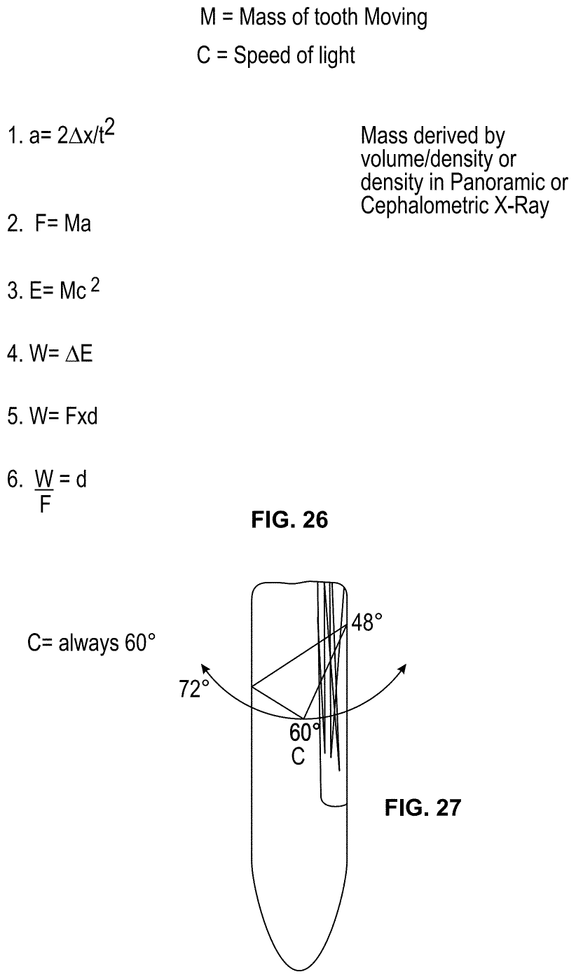

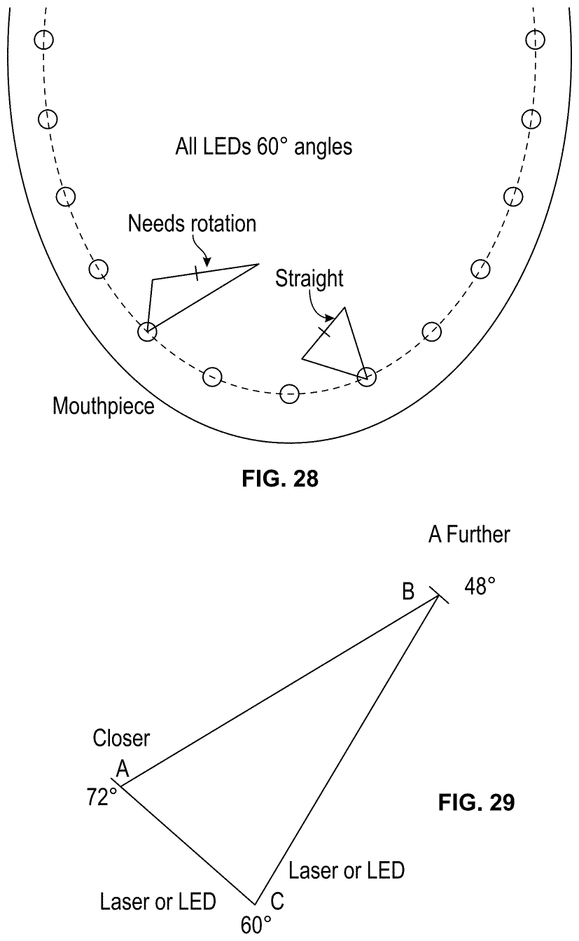

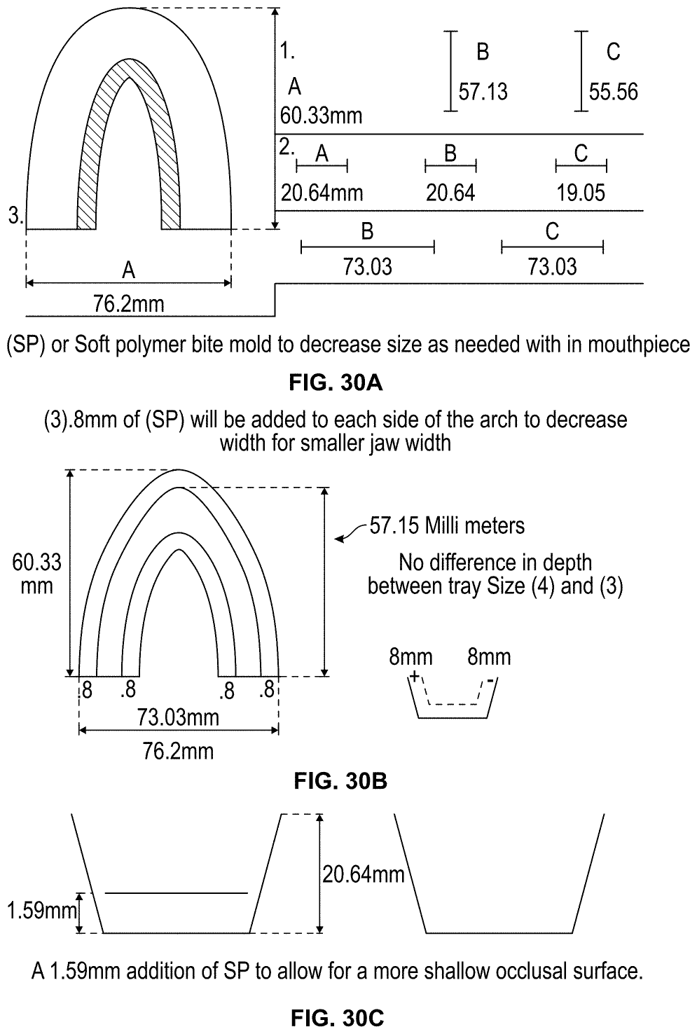

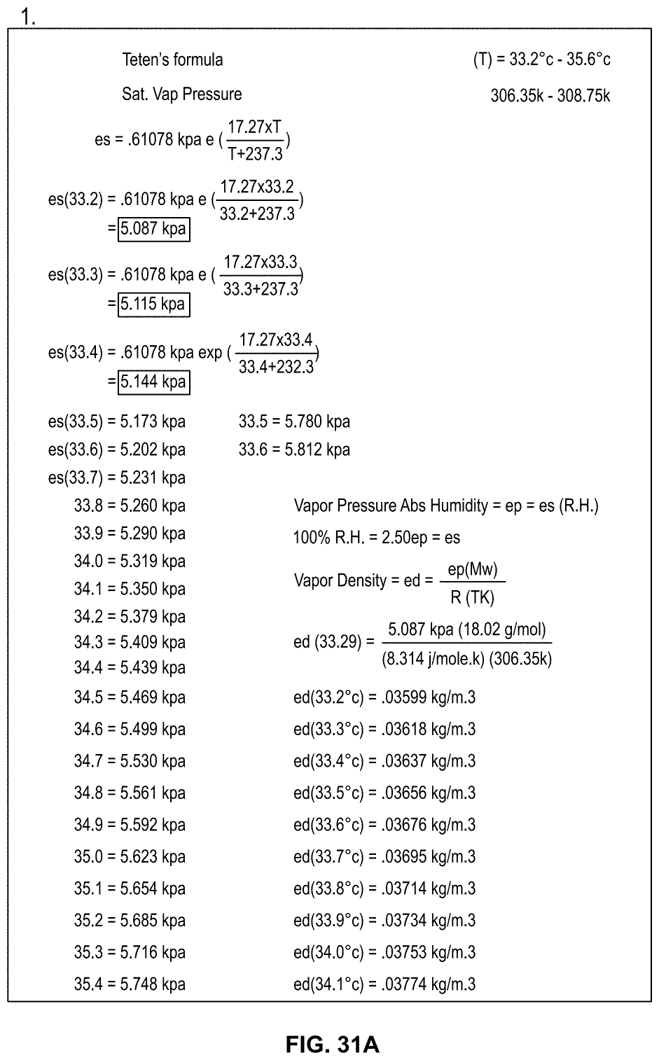

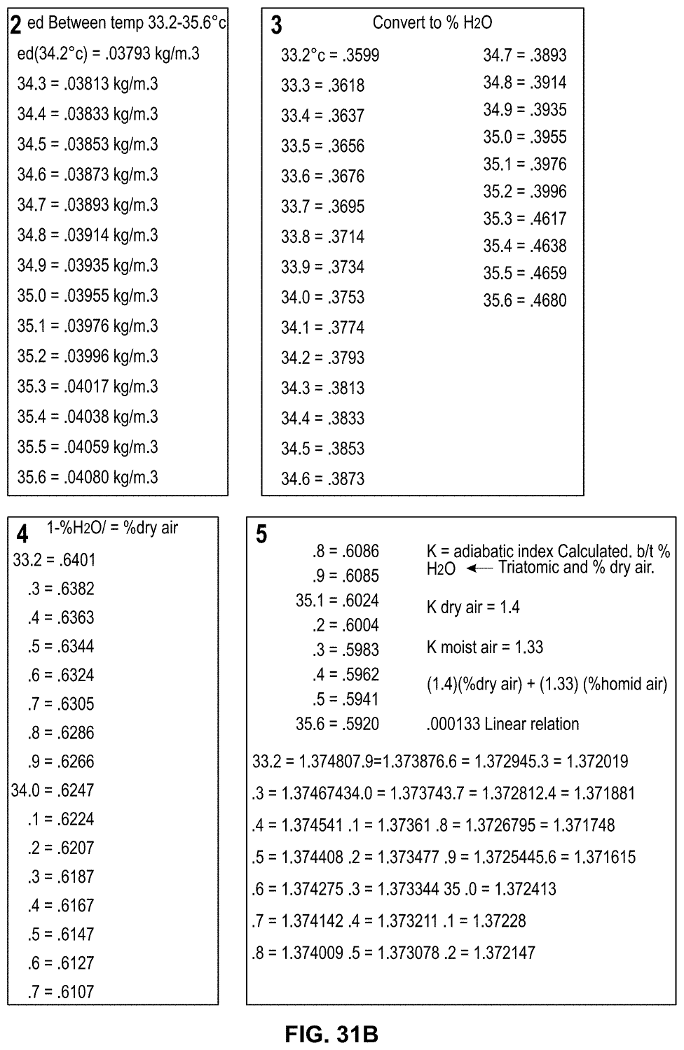

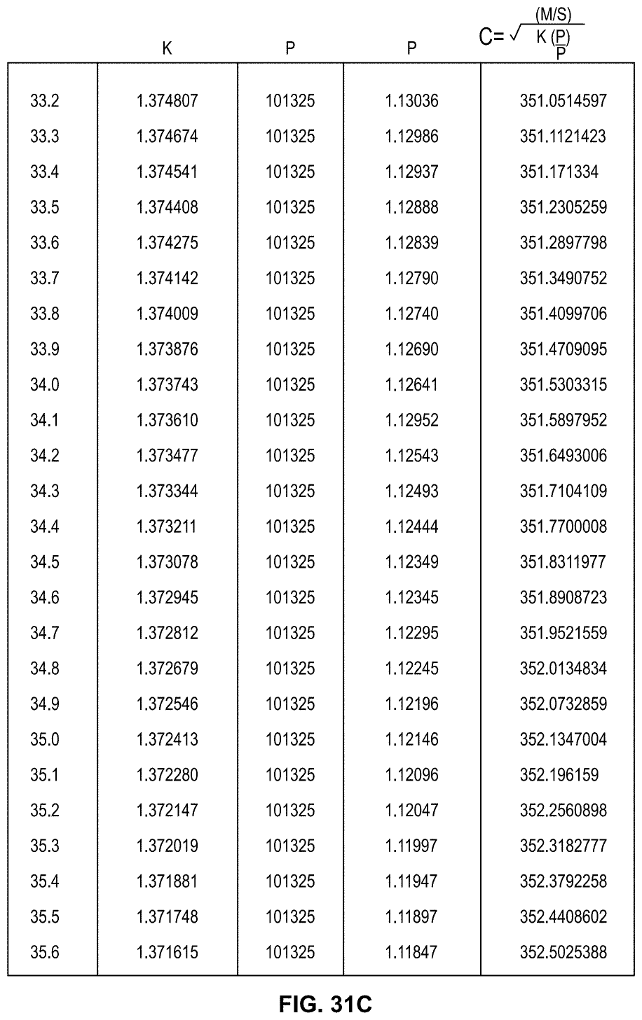

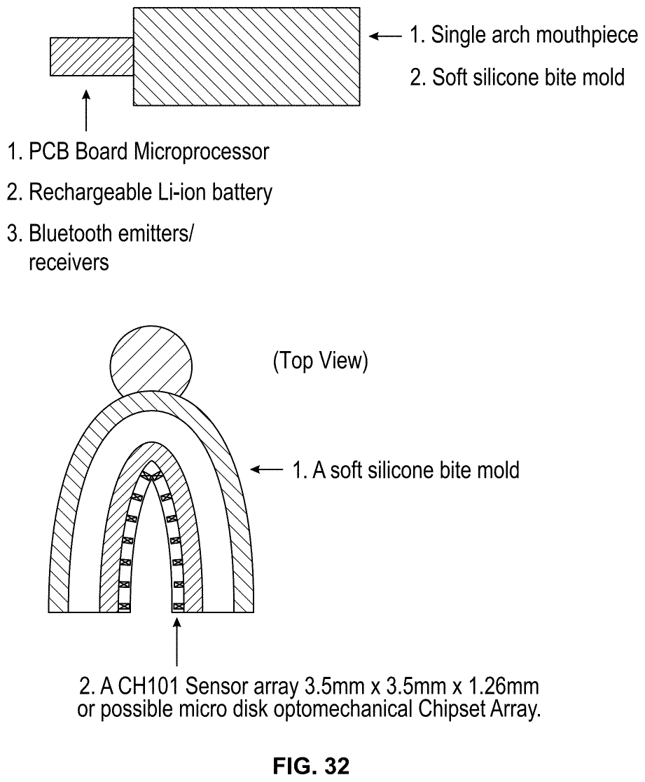

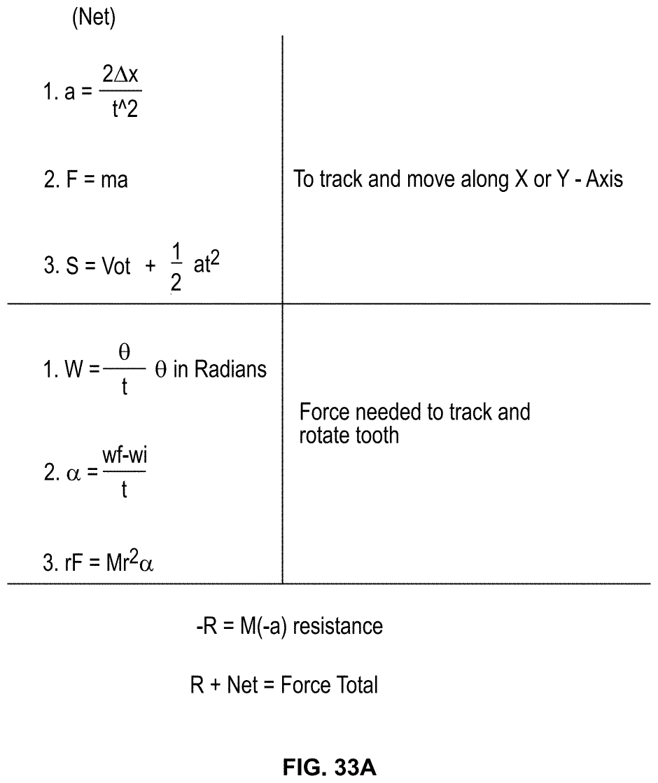

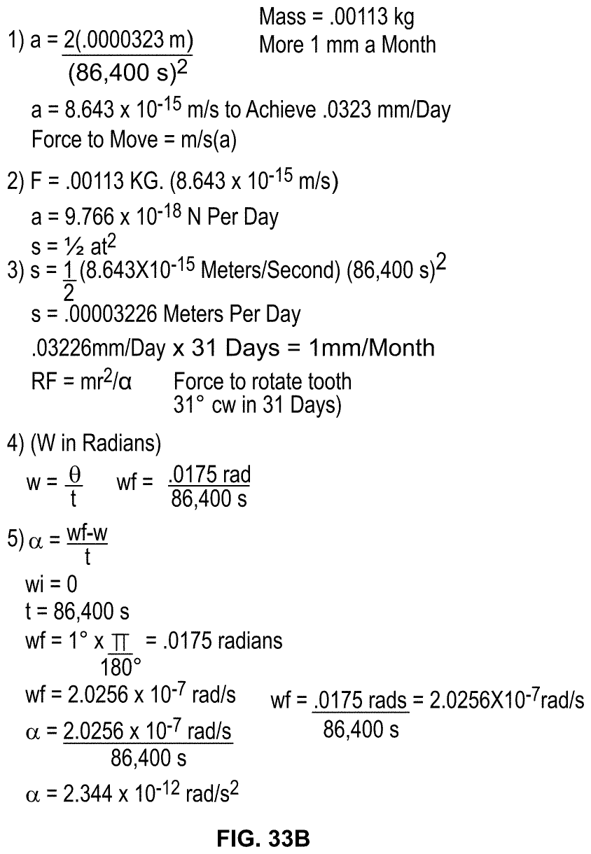

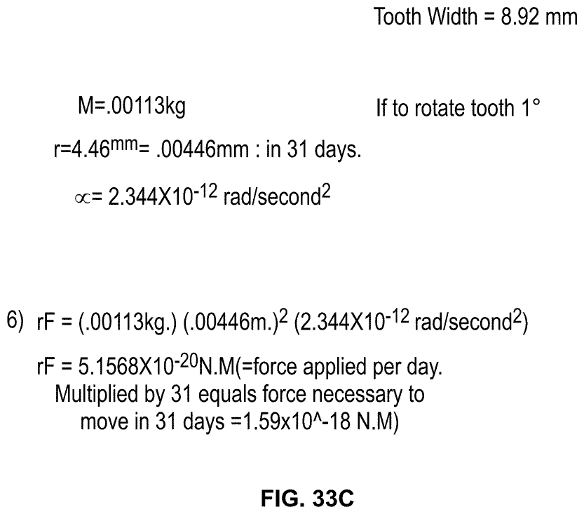

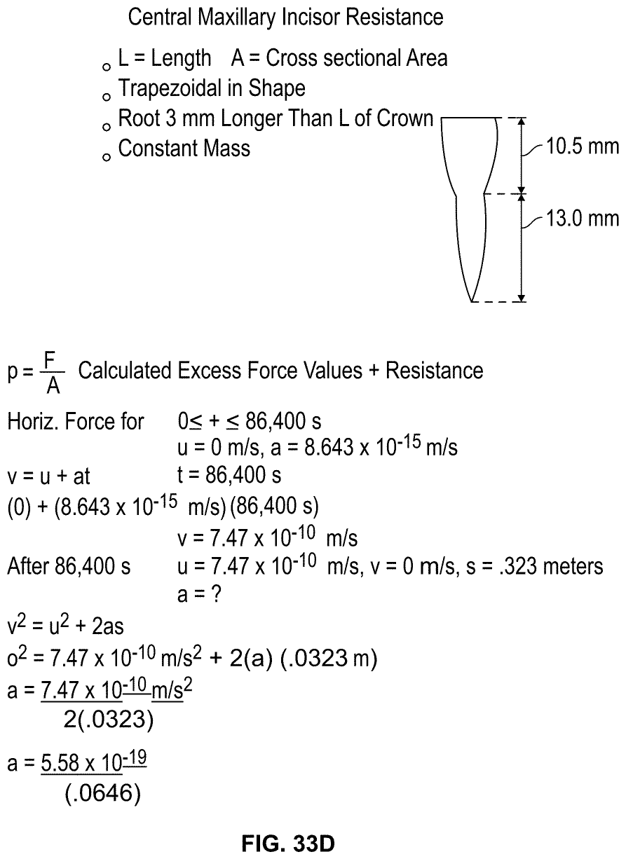

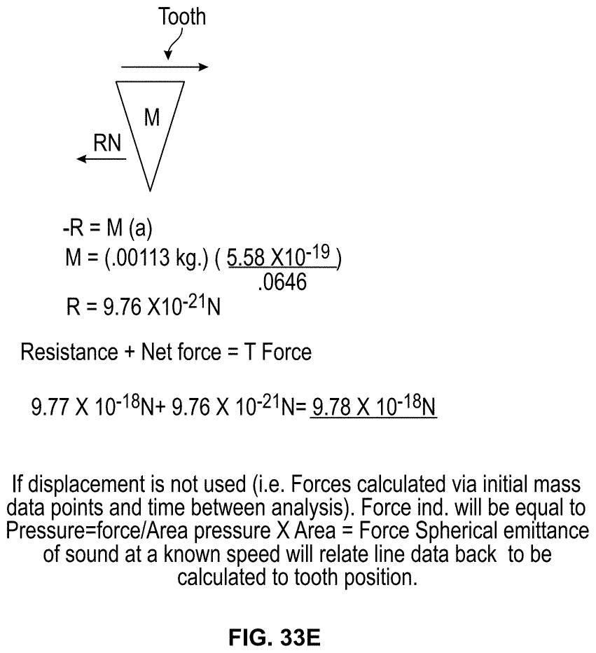

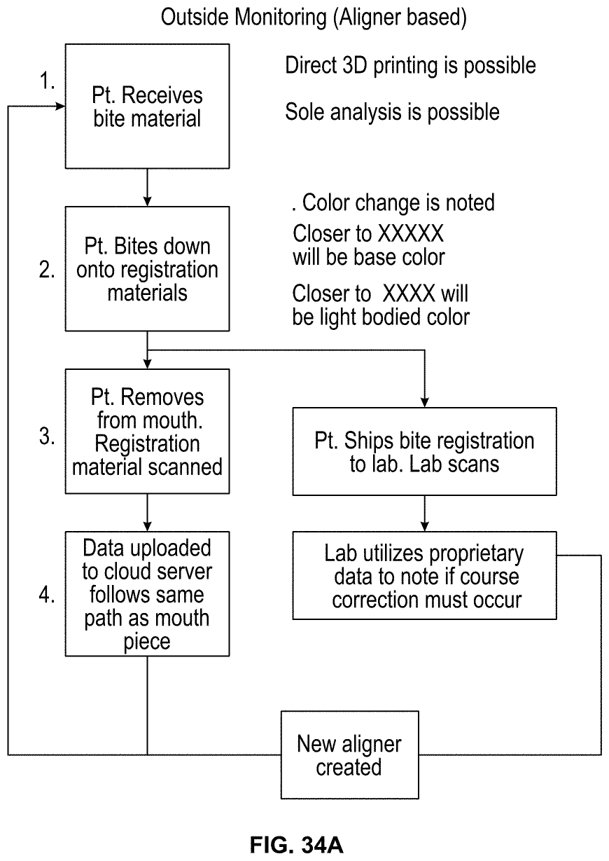

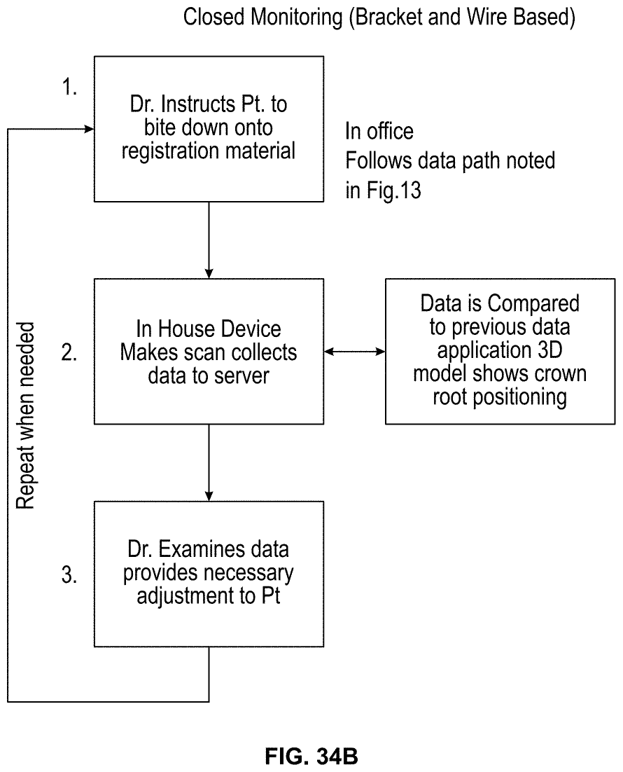

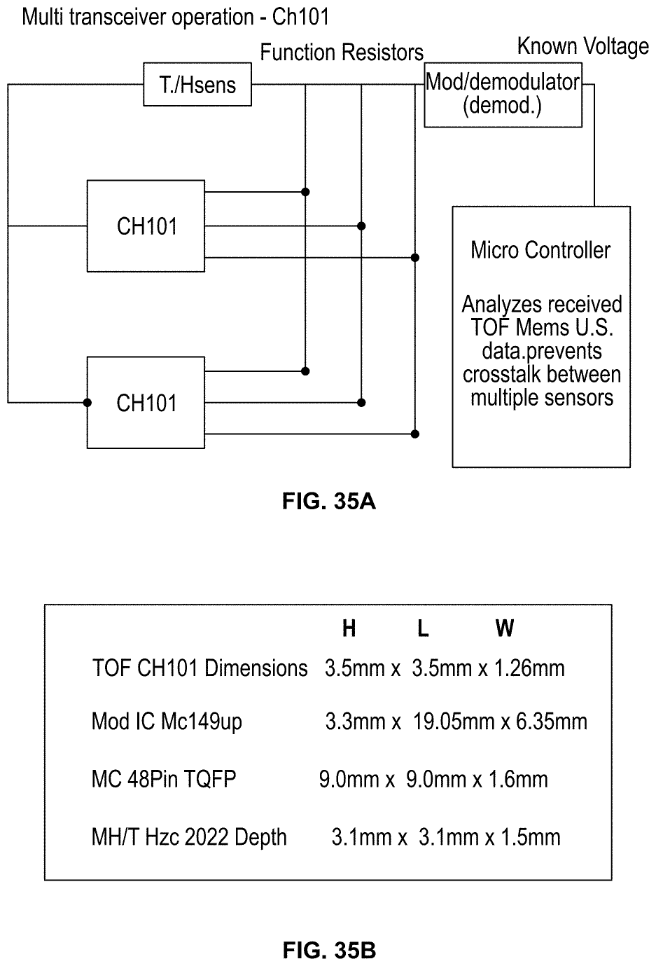

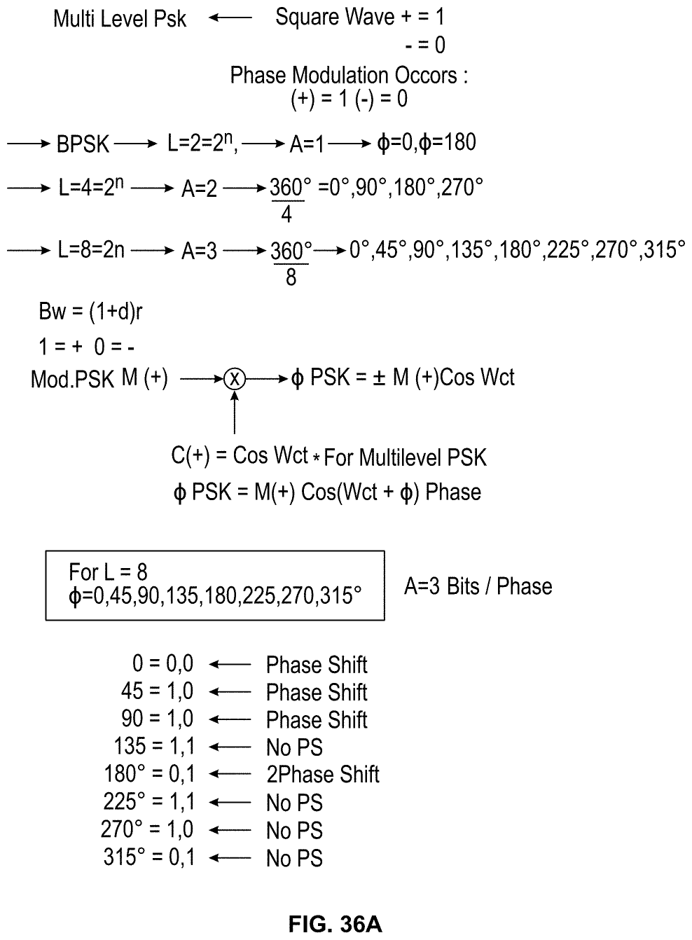

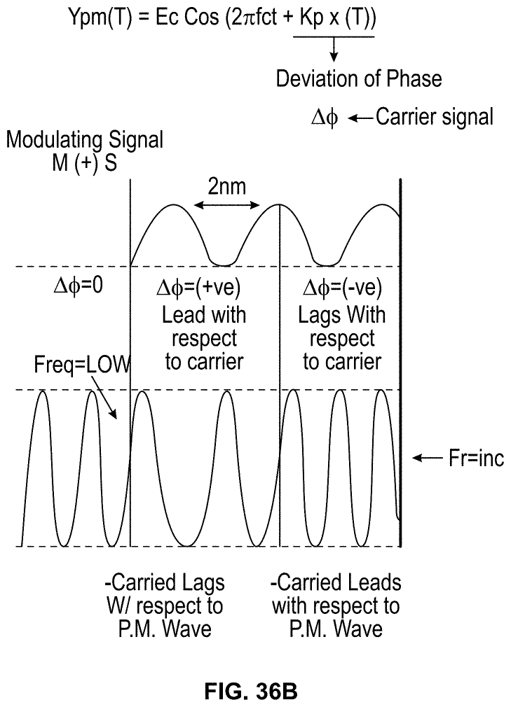

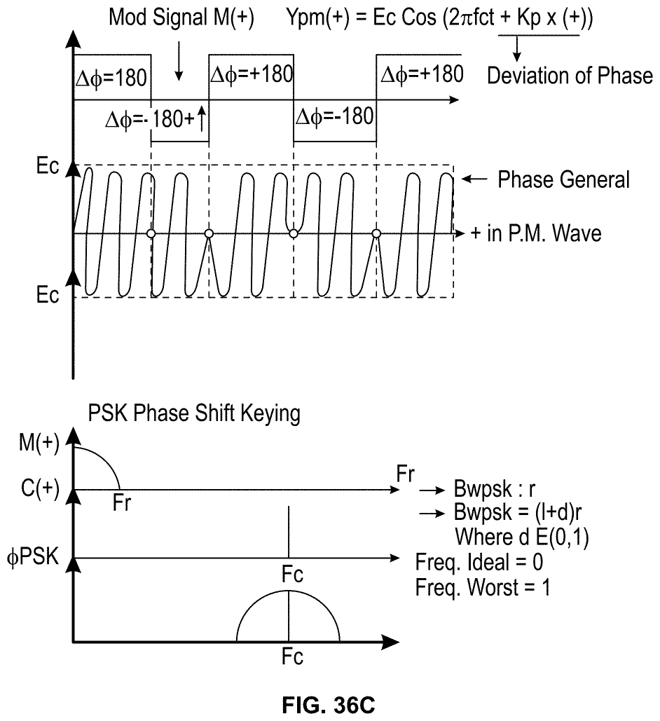

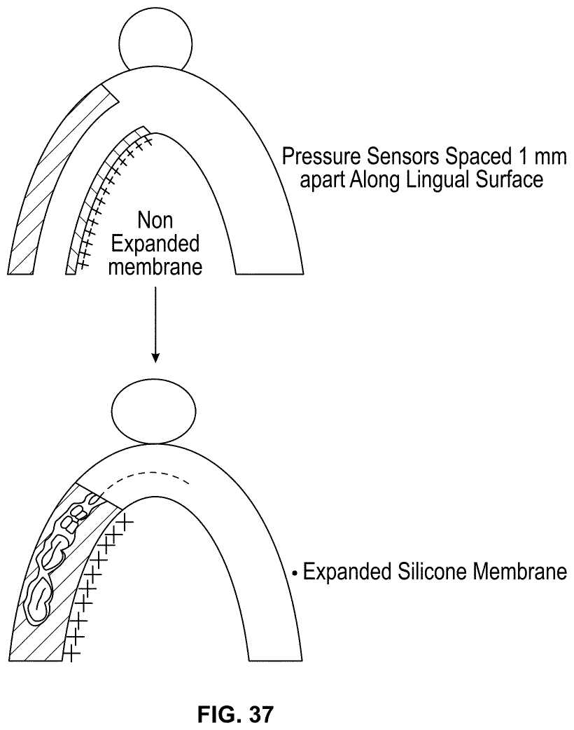

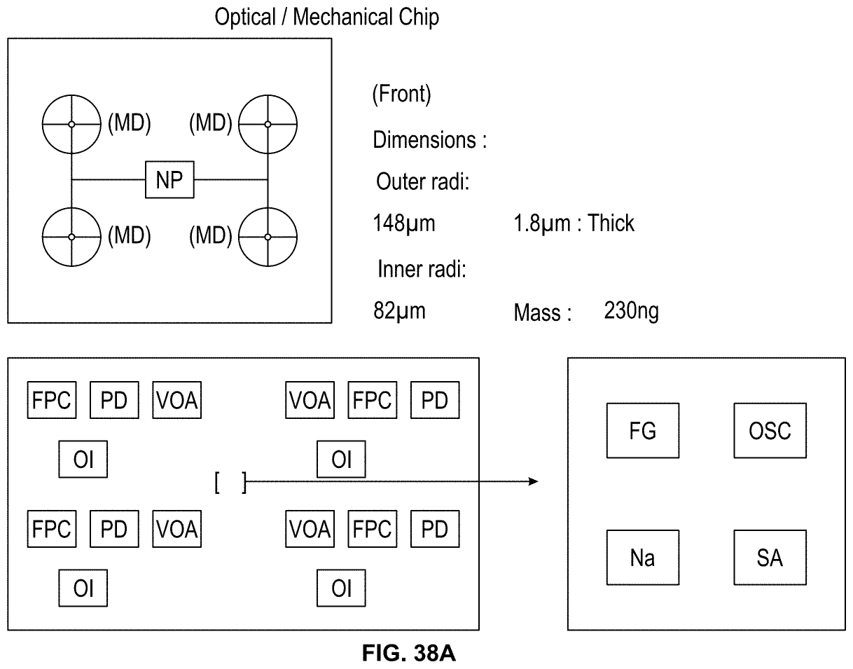

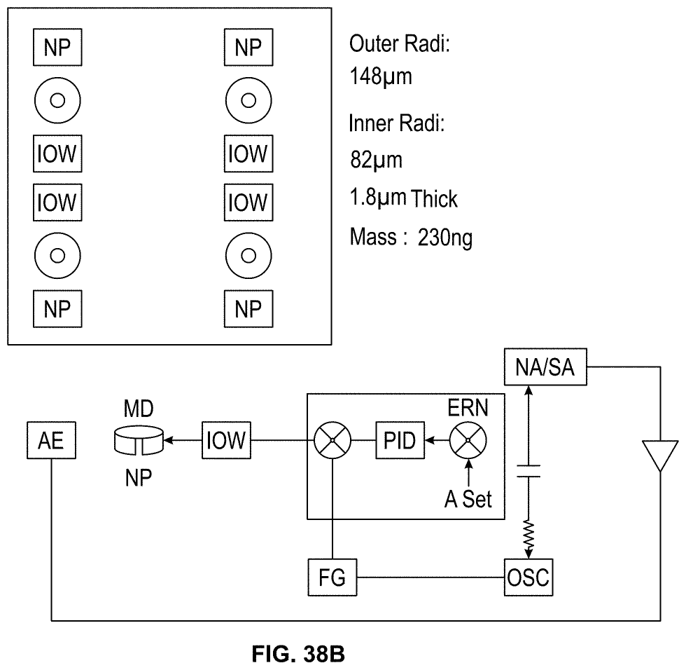

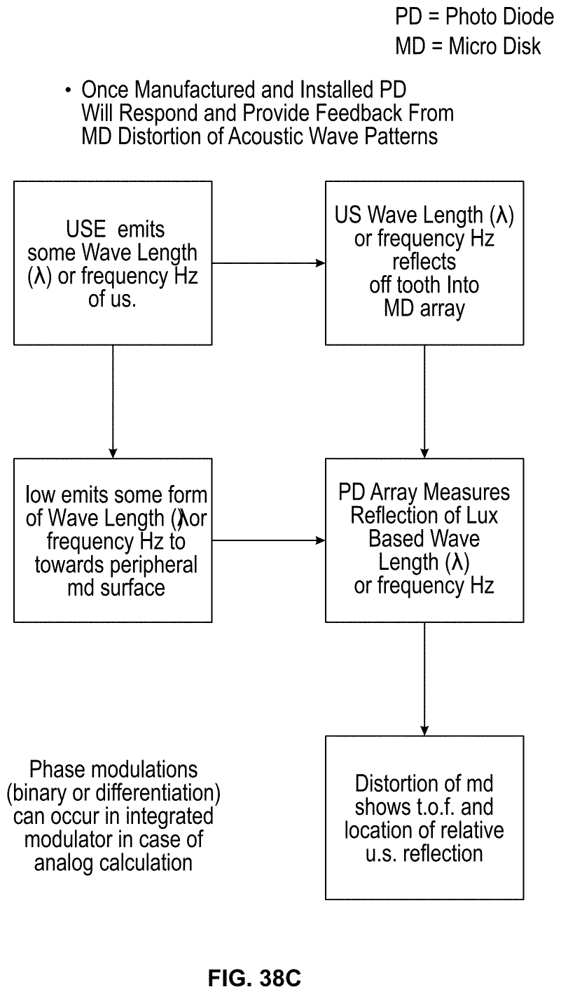

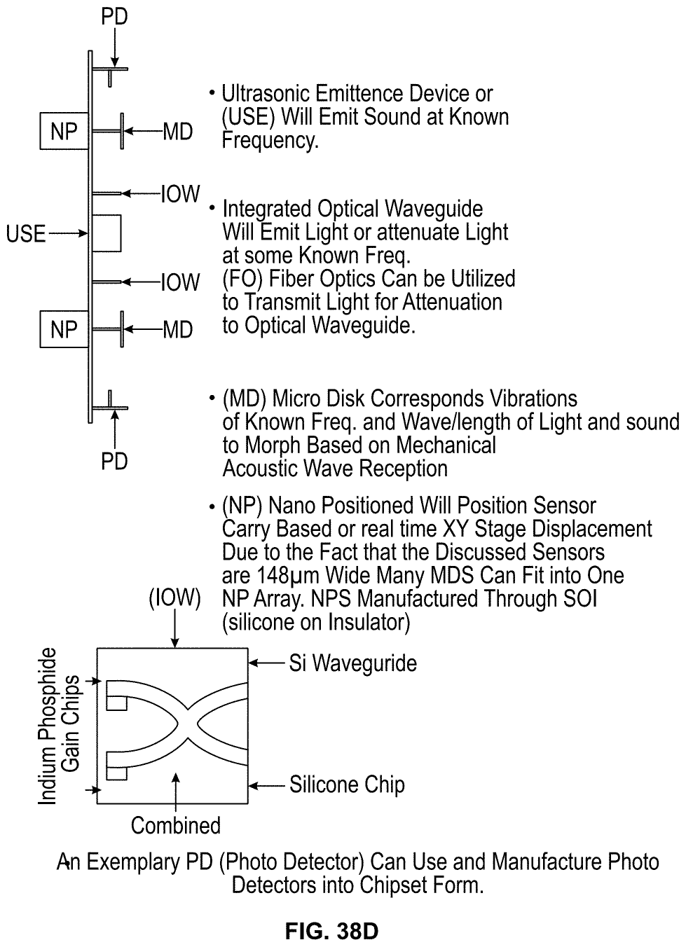

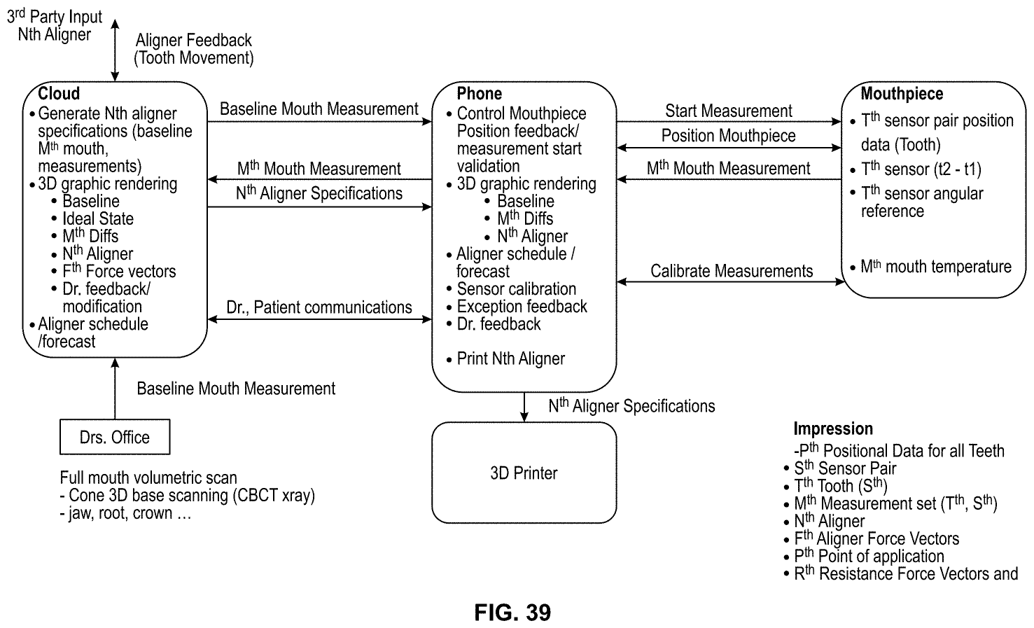

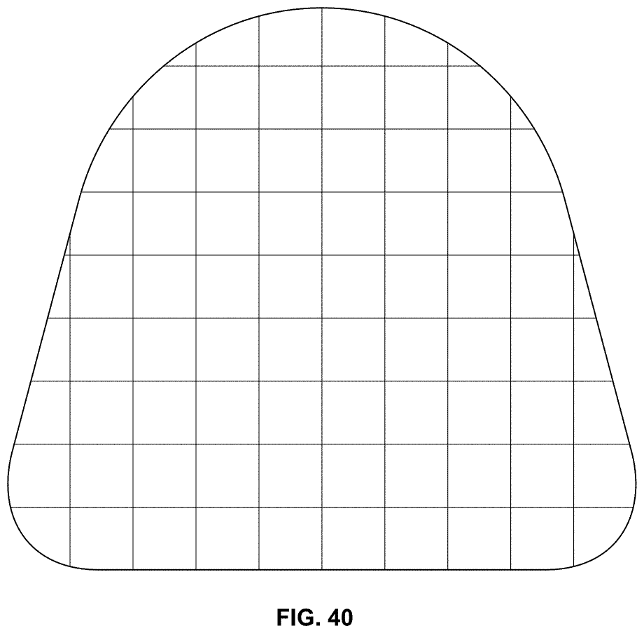

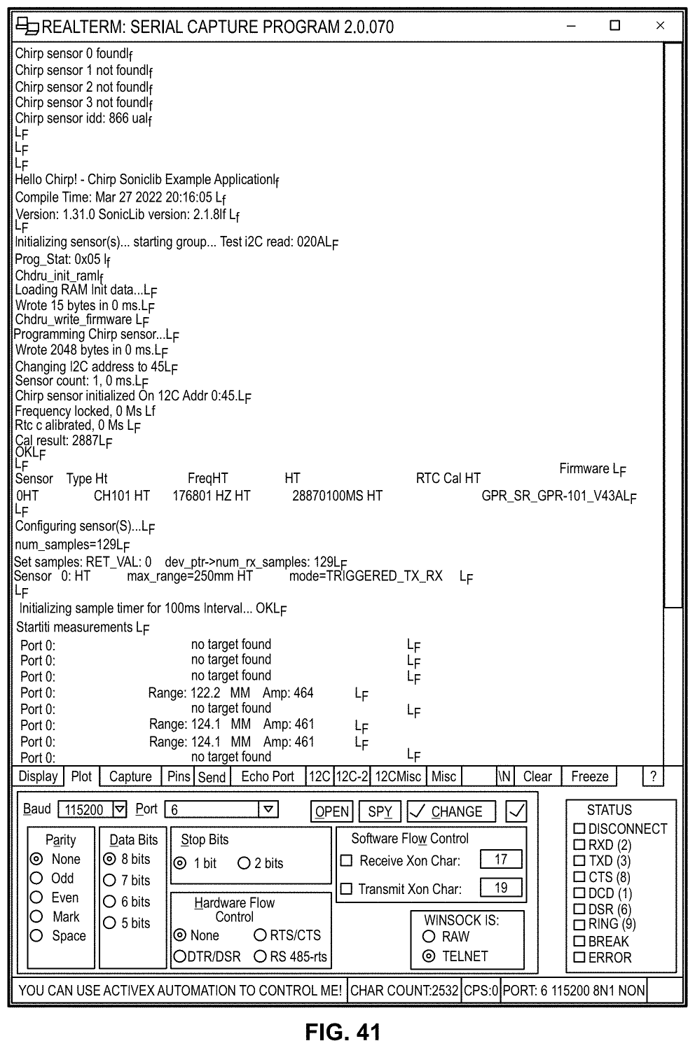

In one aspect as shown and disclosed further in the drawings, the orthodontic alignment system 10 of the present invention includes a mouthpiece 12 that has a housing body 14 forming a U-shaped channel within which to receive the patient's teeth. Although in the present embodiment, the housing body 14 is shown as two pieces 14a and 14b, it may be formed from either a single piece or several pieces. The mouthpiece 12 is designed to be used in combination with an adjustable polymeric orthodontic aligner tray 16, such as that shown in FIG. 18. A computing device 18 (e.g., FIG. 21) is operatively connected to the mouthpiece 12, either by wired or wireless connection. The mouthpiece 12 is sized appropriately to be placed in a patient's mouth and fit around the patient's teeth. The mouthpiece 12 has emission source 20 sized to be mounted in the mouthpiece 12 and configured to emit light or another emission source (e.g., sound waves) into the channel of the mouthpiece 12 and toward the patient's teeth. In one embodiment, the emission source 20 will include one or more LED light sources for each tooth position and corresponding sensors 22 for detecting light reflected from the patient's teeth. In some cases, the emission sources 20 and sensors 22 may be integrated into the same space, as shown in the present embodiment. The computing device 18 includes memory and a processor for performing calculations and processing data.

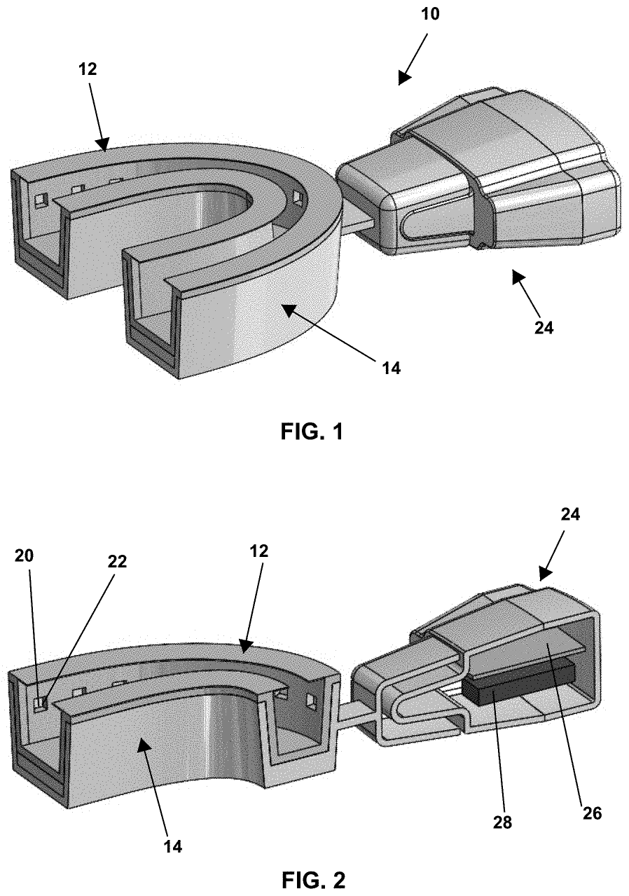

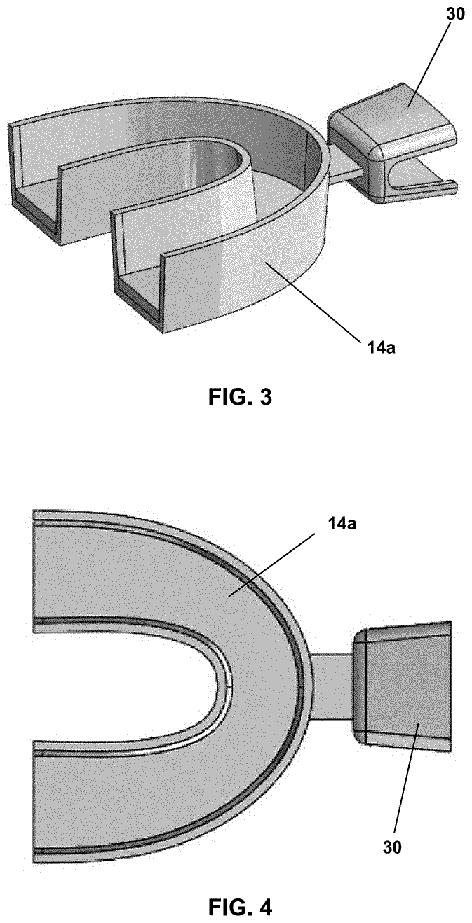

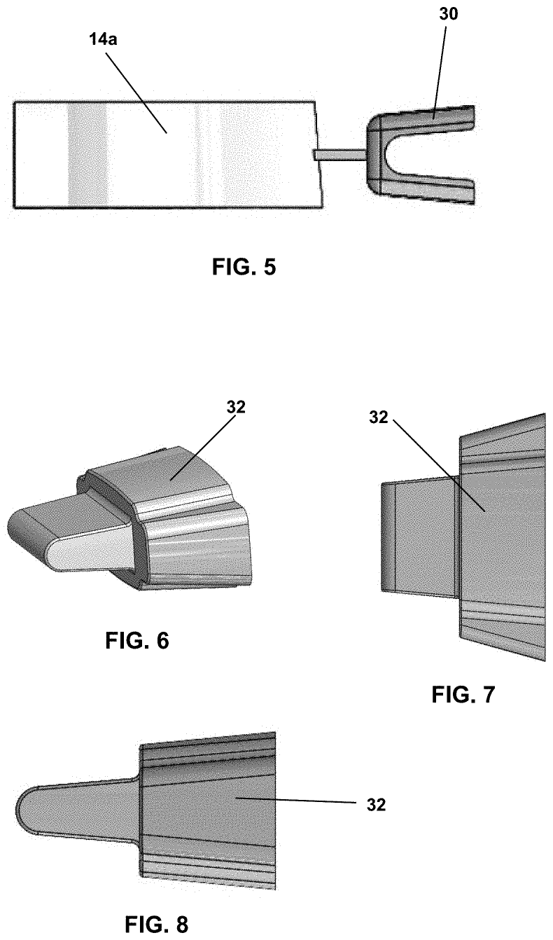

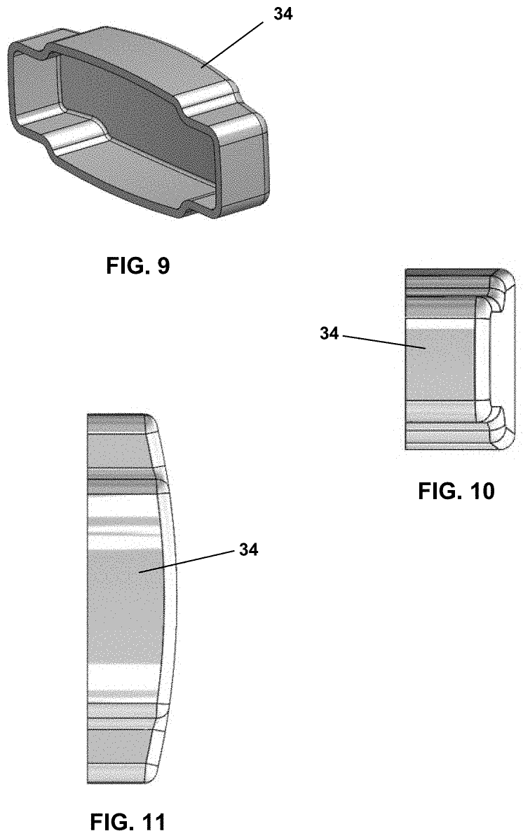

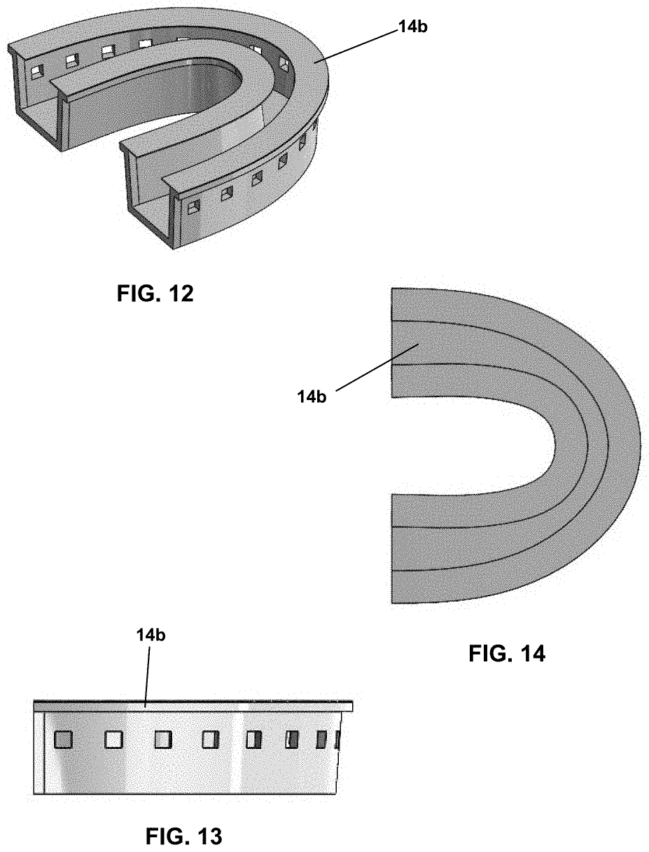

In the embodiment shown in FIGS. 1-13, the mouthpiece 12 includes an electronics housing 24 which houses a printed circuit board 26 and a battery 28. In the presently shown embodiment, the mouthpiece 12 has an integrated attachment receptacle 30 which receives the body 32 of the electronics housing 24. The presently shown embodiment also includes a housing lid 34 for closing the body 32 of the electronics housing 24. The lid 34 provides access to the circuit board 26 and battery 28. In other embodiments, the electronics housing 20 may be replaced by a wired connection from the mouthpiece 12 to the computing device 18 (e.g., personal computer, smart phone, tablet, etc.), as will be recognized by those skilled in the art.

In an alternative embodiment, the present invention has a “sleeve” that fits over or clips onto the back of any smartphone or tablet and provides light from the LED light on the phone to the mouthpiece 12 using fiber optic cables. A spectrometer or the camera itself can be used to detect light wave data which yields characteristics of tooth alignment.

In yet another embodiment, the mouthpiece 12 shall be an initial size, with the usage of soft polymers or soft silicone medical rubber to alter the size of the arch required to achieve accurate measurements and placement at each regularly scheduled adjustment. The mouthpiece 12 consists of a hard plastic portion that is the same for all mouths and a soft portion that changes in size depending on the needs of the patent. The size of the soft rubber piece will change for those with a more narrow or wider arch. The “holes” for placement of sensors will be in the same position regardless. The sensors are mounted to the inside of the plastic emitting through holes in the soft rubber. This ensures accuracy in measurement and makes manufacture uniform. The addition of said polymers will allow for a stable fitment at each weekly adjustment. These polymers will slide over the sensor arrays to allow for universal fitment and analysis constants. A mathematical correlation of current tooth alignment position vs. initial and previous data will allow for a constant of uniform fitment at each adjustment cycle. The mouthpiece 12 within this embodiment consists of a singular arch mouthpiece 12 which can be taken out and placed on the patient's maxillary and/or mandibular arches. Note that this is different from the noted singular mouthpiece 12 that measures both arches at the same time. This mouthpiece 12 would still connect to the patient's smartphone application and still send and transmit data in reference to the patient's weekly remote analysis.

The mouthpiece 12 is connected to the computing device 16 which has spectroscopic software for converting the collected spectroscopic data into information concerning the position and alignment of each tooth. Computing devices for hosting the spectroscopy application can include smartphones, tablet computers, notebook computers and the like, or can include a dedicated computing device that is specially designed for use with the present invention. The collected data regarding the position and alignment of each tooth can optionally be combined with information from a panoramic, cephalometric, cone beam scanning, or 3D X-ray to measure the root length of each tooth and detect osteopores within the patient's maxillary and mandibular arches. Using the dimensions of total length of root and crown combined with the position and alignment data collected from the mouthpiece 12, an orthodontist develops a weekly or monthly alignment plan for adjusting the patient's teeth. The mouthpiece 12 allows the orthodontist to collect data remotely can then on a week by week or monthly basis read and analyze the amount of force to be applied to the patient's mandibular and maxillary arches.

As noted, the mouthpiece 12 is paired with an aligner tray 14. The aligner tray 14 optionally utilizes retro reflective pigments, pressure sensitive polymers, or holographic films and polymers or the like. Retroreflective pigments are essentially polymers that can be developed to reflect and refract light at given angles. Pressure sensitive polymers are polymers that when exerted under force change their color or shade of color. The utilization of such pressure sensitive polymers would allow detection of change in color which is given by its relation to the visible light spectrum.

For example, if a patient has a number six canine that has erupted in an ectopic manner (i.e., the tooth is poking out) the polymer enhanced aligner would essentially give off a force indicator on that number six canine when compared to the number 7 and 5 teeth which by chance have erupted in a normal anatomic position. The aligner would give off a visible indicator (red 620 nm) when compared to the five and seven (both 495 nm). The mouthpiece 12 would essentially use white light as a constant source and in turn receive the spectroscopic analysis to the patient's smartphone application. The application would store the data, thus allowing the orthodontist to view this patient's treatment progress from start to finish without ever seeing the patient. This prevents any and all periodontal related damage, making insurance-based filing fraud virtually impossible to commit. It also allows tracking alignment of each tooth or the entirety of all teeth on a given arch down to the Newton of force applied, yielding much greater predictability and maximizing treatment efficiency.

### Relation of Newtonian Physics to Treatment Process and Methodology

The displacement of the tooth or plurality of teeth (i.e., the calculation of the speed of light over the time taken to emit and receive light divided by the time between analysis cycles) follows the same basic principles regardless of which of these methods of determining tooth position are employed (light, sound, radio frequency, or the like). The wavelength intensity and amplitude of the emitted light will be constant, while the intensity or amplitude of the reflected light will differ between adjustments because the teeth have moved (this method relying on reflection intensity). All other Time-of-Flight, “TOF,” forms (light, sound, radio frequency or any form of electromagnetic spectrum) are solely dependent on the time taken from emission to reception, i.e., the distance calculated due to the relative speed of each emission method. The information received is stored along with root and crown length data. Velocity is calculated using the equation force equals the change in distance of the respective teeth divided by the change in time taken for those respective teeth to move to position. Velocity is calculated using the change in position “x,” (the final position minus the initial position) divided by the time taken to move the tooth. Acceleration of each concurrent tooth is calculated as two times the change in desired position of the tooth divided by the total time between analysis cycles squared. Mass of a tooth is calculated via volume of the respective tooth multiplied by the density of the tooth. Volume of the tooth can be determined from the patient's panoramic, cephalometric, CBCT X-ray, or irregular shape volumetric computation as discussed herein. This form of computational volumetric analysis is made at the start of treatment. The practitioner then decides on the optimal distance to move or adjust the patient's dentition on a monthly basis.

The listed Newtonian physics formulas related to force acceleration and mass are used to suggest the most optimal and efficient way to move the teeth into their respective positions for the benefit of patient safety, e.g., 1 mm a month divided by 31 days is equal to 0.0323 meters per day (two times the change in position X divided by time squared equals the acceleration of said tooth within a 31-day period.) The mentioned methods of tracking displacement can be used on a weekly basis to analyze these predicted variables on the patient and allow changes to be made accordingly. Since acceleration is determined to be set upon the desired and suggested movement, force in Newtons can be calculated dependent upon predetermined mass of the tooth which, unless due to physical trauma, will remain constant throughout the entirety of treatment. The inclusion of the kinematics equation for displacement (displacement equals final velocity multiplied by time plus one half multiplied by acceleration and time squared) allows the entire system to verify itself over the course of treatment and can catch motility errors and or lack of patient cooperation with the utilization of the inventive methods disclosed herein.

In another embodiment the usage of mechanical sound TOF sensors will allow for a form of detection with the noted exemplary speed of sound within the mouth. The currently disclosed method of detection of movement remotely will apply for both electromagnetic and mechanical sound speed. The sensors will be placed in known locations on both or either the lingual or buccal sides of the mouthpiece's interior surface. The method of applying only these sensors to the lingual or buccal side of the interior mouthpiece 12 follows the concept of the symmetry in relation to the width and size of the mesial and distal sides of teeth. The concurrent monitoring of one side of the tooth shall follow the notion that tooth movement is related to all aspects of the tooth and root (i.e., movement of the rear face of the tooth will directly correlate with movement of the front face and root of said tooth). This method follows the laws of mechanical wave emission, which states that emission of sound waves occurs in a 3D spherical form (area of spherical wave 4(pi)(r\hat{ }2)) from point following the further reception of said waves which would allow complete image construction with a plurality of Ultrasonic and or sound based TOF sensors.

### Full 3D CAD Model Development Methodology (Direct Imaging)

One such application of the present invention is to decrease the cost and drastically increase the availability of full mouth intra-oral scans. Utilization of ultrasound, sound, or photon (light) based TOF sensors can be used in a plurality to develop full intra-oral scans of the dentition (Note: this is different in application than the utilization of said sensors for TOF to determine displacement of the dentition or distance between emission points and reception calculated via time). The utilization of Hamiltonian coding within off the shelf TOF products will both increase resolution and decrease error within the development of images based on spherical wave emission and concurrent reception. Most if not all off the shelf products utilize modulation and demodulation of said waves as either sinusoidal or square waves. Sinusoidal waves in TOF imaging and sensing report higher amounts of error because they are not received as perfectly sinusoidal. It shall be noted that the spherical emission of sound waves has application in determining the shape and curvature of teeth, in addition to its use to determining displacement of a given tooth. This method of direct imaging is more applicable and cost effective in a remote form as the price for such units will be much cheaper than the fifty-thousand-dollar list price for in office technology utilized to gather CAD models of the dentition (iTero and the like), potentially allowing the technology to be distributed to the patient remotely.

For calibration purposes and uniformity, the smartphone application will preferably have an integrated reference chart which displays the speed of sound between a range of 33.2 and 35.6 degrees Celsius at one-hundred percent relative humidity. The mouthpiece 12 will have some form of temperature measuring diode or device to record ambient temperature that allows the device and application to instantaneously realize the speed of sound in said environment. Alternatively, calibration be accomplished by using a sensor independent of all other sensors set at a known distance to one point within the mouthpiece 12, or oral cavity. The time between emission and reception will allow for exact speed variables to be known regardless of temperature and humidity.

In one embodiment, the tooth surface itself acts as a surface layer. A first layer of the aligner tray 16 acts as a “secondary layer” and preferably includes extremely small reflective beads. Orientation does not matter from varying monthly aligners as long as the emitted light source and received light source are equidistant from the tooth surface. A second aligner layer acts as the catalyst of force and relief, physically adjusting teeth in the manner prescribed by the orthodontist.

### Broad Application

The present invention allows for the development of aligners that utilize the aspects of the present technology in combination any application that may lie within all fields of dentistry that already occur within the industry (preexisting methods including but not limited to aligners, brackets and wires, or any future methods of movement). All methods of movement or observations of dentition in all fields of dentistry can be used in pair with the mouthpiece 12 and methods of the present invention. This allows pre-existing methods the use of said technology for more accurate in person or remote treatment.

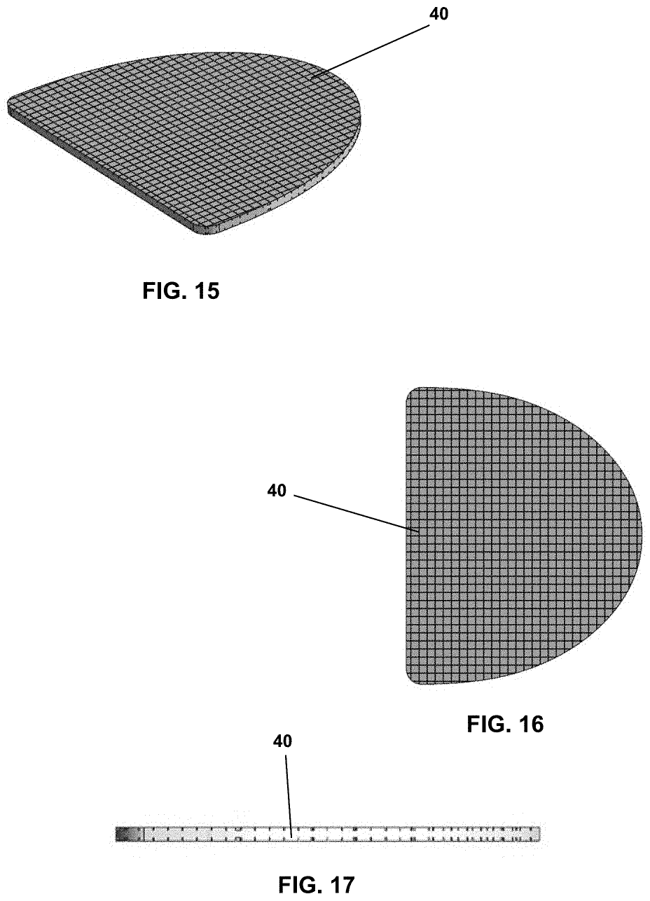

Occlusal position monitoring and its relation to positional prediction and analysis In yet another embodiment, the patient is given and or shipped some form of bite registration material 40 including, but not limited to, carbon film, polyvinyl siloxane, shim stock, or even thermo/halochromic chemical composed polymer. This material will consist of a multi layered material that includes a substrate of different base color to the color that is layered onto said substrate (e.g., top layer blue, middle layer green, lower layer blue; or a heavy bodied material affixed to a light body substrate). A preferred shape for such bite registration 40 material is shown in FIGS. 15-17.

When the patient bites down, the differentiation and melding of color will preferably show a dimensional array of occlusal relationship to bite color (e.g., deeper occlusal bite would be green). The general idea is that as teeth move to desired positions the occlusal relationships between the maxillary and mandibular arches will show an increased and gradually aligned relationship. The determination of this occlusal relationship will allow full analysis of said dentition (root, crown, all inclusive). At the initial appointment a full mouth intraoral scan and/or CBCT x-ray with cephalometric x-ray or panoramic x-ray would be given to said patient. The relationship between the cephalometric or panoramic x-ray and full mouth crown intraoral scan is discussed below. The initial registered data will be collected within a server or cloud-based computing system, which in turn will be paired with the patient's smartphone application. The present methodology of connecting patient data to doctor will follow with the other forms of analysis mentioned within this disclosure.

At selected times, (e.g., weekly) the patient can be shipped or delivered bite registration material. The patient will then bite down and remove said material. Once registration is taken, analysis of registration can be achieved via a device which uses spectral analysis of differentiating color to show depth of bite and level of alignment between the maxillary and mandibular. This device can either be a fixed unit in a locale to which the patient will ship said registration or be achieved via a device that utilizes a smartphone camera or a device which is sent to the patient to scan and upload this data to the smartphone application which is then uploaded to the cloud-based server. Once data is uploaded to the cloud-based server, algorithms will relate the occlusal mapping to the original tooth dimensions set forth at the beginning of treatment, allowing complete aligner construction with a mobile form of data collection.

The relationship between the cephalometric or panoramic x-ray and full mouth intraoral scan coincides with the full, crown-only mouth scan which will give the application some defined area of tooth surface above the gingiva. This relationship will then carry over into a defined proportional surface area relationship to the root surface area below the gingiva. This is achieved via analysis of patient's 2D x-ray when surface area is known of the crown-based scan of the tooth. A directly proportional mathematical derivation can be made towards the surface area of the root surface.

### Positional Relativity and Observation with Subsequent Method

Turning now to FIGS. 15-17, one method relates to remote occlusal bite monitoring and utilizes an approach that is similar to quick response codes (QR codes) in accessing specific data within a small space. The orthodontic approach utilizes a black grid of known dimension laid onto a white background (0.5 mm×0.5 mm square exemplary). The patient information and data flow remain the same (i.e., patient receives pretreatment scans in office). However, instead of a mouthpiece 12 the patient bites down onto a substrate (wax, PVS material) which in hand has an indicative black line grid laid onto it (wax, carbon paper, BPA/BPS/Phenol free thermal paper). At each cycle and with the concurrent intentional movement of the patient's dentition, deformities of aforementioned gridlines will show relativity to teeth on both the mesial and distal side and those teeth which are relative on the opposing jaw. Due to the fact that the patient's data within the smartphone application contains an initial form of accurate 3D model data, the patient's bite alone can relay relative position data into the same smartphone application via a grid and/or known dimensional analysis.

The method relies on PDF file scans of noted dimensional substrates (occlusal markings) with the patient's smartphone. Once the smartphone receives a PDF file of the noted substrate, it is then converted into a .svg/SVG file, which takes a pixelated image and then converts the black gridline into vectors via mathematical formulation on points or lines on a grid. If necessary for relation to the software-based 3D model, it can then be converted into a (DWG/.dwg) file, which in turn allows vectors and distorted changes on the grid to be immediately related to tooth position within the smartphone application. It shall be noted that due to the standard grid size on the wax bite a CAD file conversion may not even be necessary to successfully track deformations on a grid; this is due to svg file types which are already predisposed to calculate such things. Once calculations occur within the PDF to SVG conversion the patient's 3D model can be altered as an accurate representation of the patient's dentition. If this utilization results in the immediate and direct 3D printing of a patient's aligner (i.e., it is used for remote analysis and/or directed tooth movement via new aligner construction), the initial volumetric analysis will serve as a blueprint for each subsequently constructed aligner. The change in relative position of the occlusal surface will show where the teeth are located at each requested time domain (adjustment cycle). The noted changes in occlusal locations and the aforementioned methodology can either be used for the sole purpose of remote tracking and prediction (via altering of CAD file and predetermined mass variables within software or application), or for the construction of future aligners based on tracked movement of the dentition within a set time variable. This method is very appealing in tracking displacement as it is extremely accurate and cost effective there is no need for associated sensors or electronics besides the utilization of a patient's smartphone, or other tool for analysis.

After each subsequent scan, either the aligners can be printed based on the gathering of this data in a physical office or building and then delivered to the patient, or they can be printed within the patient's residence with a supplied 3D printer via fused deposition modeling. A ‘standard’ may be necessary to create at the time of the patient's introductory appointment not only for calibration, but also to familiarize the patient with the process. If necessary and for more precise calculation, image processing can be utilized to rotate the .swg or DWG file types to gain perspective from any angle deemed necessary to view deformations as a complete whole.

### Utilization of Occlusal Monitoring Irrespective of Motility Calculation

All aforementioned methods of occlusal monitoring (measurement via bite registration) can be used digitally for the purpose of verification of algorithmic, intentional, or unintentional movement of the dentition. Change in position can be determined and automatically updated in the application software. For example, most aligner companies develop all sets of aligners prior to the patient starting treatment, without any way of verifying wear or proper fitment with remote accuracy throughout the course of treatment. This provides an extremely unsafe and unpredictable way to achieve tooth movement and poor aligner accuracy/predictability. With the utilization of remote occlusal tracking, which shall be held different than analysis and calculation, the patient would simply bite down onto any occlusal registration material to verify the intended movement of each subsequent aligner on the same date that they are scheduled to change aligners.

### Method of Color/Frequency and or Wavelength Grouping to Determine Occlusal Position

Polyvinyl siloxane layers or any form of layers of wax polymer or any of the aforementioned materials of varying color used to detect occlusal bite registration can be analyzed within a smartphone application and/or imaging device based within an office. The smartphone application method relies on visual spectroscopy which can be attenuated to the color that presents itself to the occlusal surface once pressure of the dentition is detected on the biting surface. The smartphone's rear facing camera can be utilized to capture color wavelengths or frequencies that would be necessary to detect the relative position of the patient's bite. Cameras can be coded to only detect colors of known wavelengths or frequencies. For example, a blue/green/blue layered polyvinyl siloxane or wax bite would reveal green wavelengths between 495 nm-570 nm where the patient's teeth are located at each scan cycle, the camera would not detect the blue outline set at 450 nm-495 nm. Monitoring positional change via direct analysis from initial bite registration allows remote analysis of the patient's dentition via change in the 3D CAD model of the complete dentition throughout the course of treatment. Another method which is related to positional relativity from a hybridized spectroscopic analysis approach can be realized by computer vision code which groups colors of like wavelength and frequency together. For example, a PDF scan can be taken of a blue green blue layered PVS or color exposing material, and image processing can be taken to account for only levels of color with wavelengths between 495-570 nm, hence taking only image and related position from bite marks in an image.

### Usage of Infrared Radioactive Induced Aligners

In another embodiment, infrared light is used as the light source. Infrared light is produced on a spectrum that is not visible to the human eye. Thus, the pigments used in the first layer of the aligner tray 16 would be invisible to the human eye. When activated by infrared light supplied from the mouthpiece 12, however, the same read signatures would result as the previous two methods. This allows the patient to maintain the desired effect of clear aligners for aesthetic purposes during normal active daily wear while maintaining treatment efficiency and yielding all the desired readings.

### Ultraviolet Light Wave Induced Aligners

In an alternative embodiment, the light source is ultraviolet light. Ultraviolet light is already heavily prevalent in the field of orthodontics. This method allows for motility and adjustments in the outer layer of the aligner and readings of tooth alignment and force detection in the lower aligner. UV light waves penetrate the outer invisible aligner tray 16, penetrate the subsequent lower layer of the aligner tray 16, and reflect to the sensors on the mouthpiece 12 which communicate with the software application resident on the smartphone or other computing device. The orthodontist can track treatment remotely with a device that communicates with the patient's software.

### Subsection of UV

### UV Hybrid 1:

This hybrid system allows the inner layer of the aligner tray 16 to be enhanced with UV polymers that will not depolymerize (soften). It will only allow for the previously mentioned exact force readings. The outer layer allows for depolymerization (softening). This means that, when struck with the correct wavelength of UV light, the polymer softens the mouthpiece. The mouthpiece is adjusted and relieved by the doctor's orders. In order to perform this adjustment, the mouthpiece contains small, pinpointed ends to apply pressure to the aligner tray, and with the patient's bite-force the other side would be relieved naturally. After the patient's adjustment the mouthpiece emits higher UV wavelength to harden all polymers.

### UV Hybrid 2:

This method follows the same premise as the above method but uses a singular aligner to detect, adjust and relieve with the associated mouthpiece. The only change is the second layer is not needed as calculations of force, adjustments, and reliefs are calculated via IR spectroscopy. Hardening of the polymer is controlled by the frequency of UV light emitted by the mouthpiece.

### Full Band and Bracket Systems Non-Remote

This system is for patients who either choose or require a full band and bracket case. Pressure sensitive color changing polymers can be used on the front fascia of nitinol super elastic wires. These wires change torsion values based on the heat of the mouth and saliva. As the wire normally aligns the teeth in the office, at each patient's appointment the polymers will change color with applied force and tension. This means the distance of each tooth can be calculated. By pairing this with a simple four dot method at the apex of each corner of the orthodontic bracket, the Y and Z axis values can be calculated as each tooth, or all respective teeth, move. If the four dots consist of halo-chromic pigments, thermo-chromic pigments, or both, one can also calculate pH and temperature of the patient's mouth by either the doctor's eyesight and noted color change or the mouthpiece's calculations and variances in wavelength.

Insurance filing can also be linked with the smartphone app along with the patient's billing information. Panoramic X-rays and/or cephalometric X-rays would also be included in the patient's private file on encrypted servers. As far as treatment predictability, all patient biometric data would be pooled, and cases with similar instances and outcomes (bone densities, tooth size, arch width, and hyalinization rate) are compiled, allowing algorithmic suggestions to assist the orthodontist. Newton force to the maxillary and mandibular (jawbones) are calculated and noted at each adjustment cycle. Competitors do not use this technology, giving them a mere seventy percent predictability. These are measurements the human eye cannot detect. All of these allow for an ultimatum of insurance companies being satisfied with the virtual impossibility of insurance fraud in filing from the orthodontist. The orthodontist or corporation will be satisfied in treatment efficiencies and margins of treatment cost and overhead. In summary, this system makes everyone happy from patient, orthodontist, insurance company, and the company selling this product.

Dental caries or cavities, more commonly referred to as tooth decay, are linked to patient genetics and other variables such as temperature and acidity. With the incorporation of thermo-chromic (temperature color changing pigments), and halo-chromic, acid-assisted color changing pigments, the patient's biometrics could also include variables such as current temperature and pH of the mouth while in aligner treatment. If all of this data is calculated for each patient, again information can be pooled, and dental cavity likelihood can be tracked. This method could also detect salivary pH, indicating whether or not the patient has undergone tobacco use or an overabundance of soda consumption.

### Piezoelectric Incorporation

With the addition of either the first layer visible light IR or UV light induced polymer, the first mouthpiece will contain UV, IR or visible light sources as well as a spectrometer in the form of a receptor to analyze the frequency of wavelengths reflected off of the tooth surface (sides from all angles), or the speed of light over time between adjustments or time taken from emission to reflection. This information will be sent to the smartphone or IPAD application for analysis by any certified orthodontist, all treatment records will be automatically recorded, and billing information will be used. All patients may have only their biometric data (tooth size, shape, and density) shared to the algorithm (no personal information), which will ultimately make the system smarter the more that it is used.

In some embodiments, a second mouthpiece 12b, FIGS. 19 and 20, will contain piezoelectric linear force vectors or micro-DC stepper motors and non-piezo electronics (actuators, pistons, cylinders, spheres) and UV emitting diodes and spectrometers to calculate force applied to each tooth over time (generally indicated by “+” in FIGS. 19 and 20). The UV light can depolymerize (soften) the aligner for the necessary application of pressure points and relief spots and then hardened into the desired shape. The orthodontist has remote access to all biometric data, which with the patient's approval will be shared to the treatment algorithm. Both mouthpieces will contain all necessary safety switches, such as bite force detection sensors, which prevents the mouthpiece from operating unless it is in the mouth and all bite force detection sensors are activated under pressure.

Piezoelectricity can also be employed in a method of sensing and analyzing the patient's complete dentition. A piezoelectric sensor can be fixed into an array similar to the ultrasonic TOF sensors. Because PZs vibrate at a known electrical input, a small chip-based resonance device can be created to measure the time taken to receive the sound waves back to a chip-based resonance device.

### Precision based Solenoid and or DC Stepper Motor

For the purpose of the adjustment of an aligner tray 16 (which is held separate from the methods which require development of a new aligner at each adjustment), a precision solenoid and/or DC stepper motor can be utilized. This method allows threads to be drilled on the X axis of a 1 mm×1 mm micro solenoid directly attached to the threads on a micro-DC stepper motor. An increase in voltage directed at the DC stepper motor will relocate the micro solenoid. Once in opportune position, the solenoid will be activated to apply a dimple of light force to the UV soften polymer-based aligner. Once the concurrent “dimple” is applied, the aligner will then be attenuated via UV frequency/wavelength to a level of stiffness which can continuously be worn by the patient for any extended length of time, or until the next adjustment cycle.

### Patient's Normal Adjustment Process

Patient installs the first mouthpiece 12 while connected to the orthodontist (or remotely and the information is sent to him or her). The mouthpiece analyzes frequency differences and, if none are noted, the patient continues wearing the aligner tray 16. If differences are noted and the orthodontist wishes to make an adjustment, the orthodontist can adjust the patient's aligners remotely. The mouthpiece 12b will set to the attenuated standard of depolymerization (point at which it becomes soft), and the actuators then activate to apply any necessary pressure points or relief spots. The mouthpiece 12b then lowers the attenuated wavelength to harden the aligner tray 14. Once the aligner tray 16 is adjusted, the patient continues wearing.

### Patient's Normal Adjustment Process—Singular Arch Mouthpiece

Patient places the noted mouthpiece 12 in the mandibular portion of the mouth. All calculations are taken, and the information is uploaded to the patient's smartphone application. The patient then places the same mouthpiece on the maxillary section of the mouth, following the previously noted process. Once data has been received the orthodontist or dental professional notes the change in dentition and allows the application to then directly 3D print that patient's aligner either within the orthodontist or dental office or at the patient's house with a supplied 3D printer. The application constructs the aligner based on current tooth position and the desired tooth position. It does this via variables of force retention and time taken to move the tooth in the provided physics equations and all analyzed and collected remote data.

### Use of Laws of Triangles and Angles to Find Rotation Calculations

One possible way to calculate force needed to rotate (without limitation to exact angle amounts) is to have two LEDS spaced a set angle apart from each other directing their points of emission towards the edge of the tooth for which rotation variables are being measured. With both LEDs at an equal angle (60 degrees exemplary), one can calculate the force required to rotate as all angles in a triangle consisting of the crown length on the occlusal surface and the given angle of the mouthpiece LEDs equal 180 degrees. If one edge of a tooth is closer to one of the LEDs, the angle given will be more obtuse than the angle that is further away from the LED angular configuration.

### Description of Mathematical and Scientific Analysis via Known or Calculated Constants

The present invention has application for all electromagnetic waves and is not limited to (UV visible and IR) (and/or) known speed (and/or) wavelength, frequency, or luminous intensity. The present invention also has application for all mechanical sound waves, not limited to (RF Ultrasound Infrasound) (and/or) known speed (and/or) wavelength, frequency, or decibel intensity non-binding of variables to be calculated. The present invention allows one to define and locate tooth position in reference to known emission point regardless of methodology of the application of said analysis or usage of physics equations. The usage and incorporation of both Newtonian and non-Newtonian physics equations shall be used for calculations based on the emission constants which are noted. The Newtonian equations can be adjusted using feedback from the actual positioning of the tooth as compared to the predicted positioning of the tooth using only such Newtonian equations. All calculated Force data should be considered net values (minimum force values relative to time and acceleration) these values should equal the summation of the resistance values. Resistance values shall be determined via relation of acceleration to tooth mass. Once resistance is known the length of said tooth shall be used in coordination with the torque value to determine the force being applied to the root in its known form. Length and width of said tooth shall be calculated via either proportional measurement in cephalometric, panoramic X-ray, CBCT scan, or via initial full mouth scan. Volume shall be calculated via length of tooth from crown to root, width from mesial to distal at the apex, and height from buccal to lingual at said apex.

In reference to the usage of Newtonian physics, the level of hyalinization and de-hyalinization of the PDL (Periodontal ligament) and its variance between each individual person or tooth, shall not be necessary to calculate as the metric and method being used is dependent upon the displacement of each individual tooth's mass and location. However, in future renditions of said mouthpiece 12 it may be possible and beneficial to use the properties of ultrasonic imaging and frequency response to said soft tissue PDL to calculate level of hyalinization and de-hyalinization (hardening and or softening of the PDL). The previously mentioned statement follows the notion that PDL is composed of a soft tissue, the hyalinization (hardening) of said soft tissue will show some difference in image or frequency of ultrasonic frequency or wave received similar to methodology used in ultrasonic imaging of the unborn fetus. As the PDL hardens the received signals will allow calculations of the occurrence of said events. Noted as well the addition of the equation rF=mr\hat{ }2(alpha) (Alpha being angular acceleration). This shall be used to calculate the level or rotational force (torque) applied in the units of Newton*Meter.

### Density Calculated Via Standard in Movement of Tooth Type

This method will relate to the way in which dust particles are calculated as having terminal velocities calculated regardless of size, shape, or density. The International standardization organization states that “. . . However, in referring to particle size of airborne dust, the term “particle diameter” alone is an oversimplification, since the geometric size of a particle does not fully explain how it behaves in its airborne state. Therefore, the most appropriate measure of particle size, for most occupational hygiene situations, is particle aerodynamic diameter, defined as “the diameter of a hypothetical sphere of density 1 g/cm\hat{ }3 having the same terminal settling velocity in calm air as the particle in question regardless of its geometric size, shape and true density.”

Dust particles occur in many different sizes and shapes all of which have different densities and behave in different ways once aerosolized. This method of applying a constant or relative diameter and density standard to the many different types of dust particles is the most accepted constant form of measurement for such needs within the industry. In this way a standard can be created in tooth movement and intentional displacement, for example, an upper canine tooth (6 and 11) in one patient can be related directly to the canine of another patient within that canine's range in size/Volume/Mass. Categorization will be necessary into three major sizes of respective tooth type (type meaning incisor, canine/cuspid, premolar/bicuspid, molar.) size categorization respective into types A (small) B (medium) C (Large). in this way irrespective of tooth mass and or newtonian physics calculations data can be collected and realized to achieve accuracy in intentional movement. Once collected data can be processed via Bell shaped curve standard deviation statistical analysis, Artificial intelligence, or the like. All calculation methods listed within this Intellectual property can be held as independent of one another or used in direct correlation with one another, meaning that, those same statistical analysis methods can be utilized in the categorization of data collected via Newtonian or non-Newtonian physics calculation respective towards displacement.

### Sensor Placement and Methodology

Ultrasonic, infrasonic and or mechanical wave sensors shall be used in part for the accurate analysis and location of the patient's dentition at each analysis session. The addition of a necessary temperature and/or relative humidity sensor Texas instruments H2C2022DEPR exemplary can be utilized to allow for accurate T.O.F. sensor readings in an analog form. It goes without mention that the utilization of relative humidity calculations can be held within two separate integrated circuit devices if deemed necessary. The exemplary use of the CH101 sensor will be described, but this should not limit the physical construction or description of the sensor technology. The usage of the CH101 ultrasonic sensor shall be placed on the lingual surface (facing the lingual side of the teeth). It shall be noted that the data sheet describes that these sensors are not applicable to methods below 4 cm in distance. That is assuming full wave cycle monitoring. With a frequency of 175 Khz and a wave speed at 351-352 meters per second. To achieve accurate monitoring via this sensor set, the usage of multilevel binary phase key shifting/ Differential phase key shifting. DPSK relying on the lack of reference to the phase carrier wave. This modulation and subsequent demodulation can be made possible with an exemplary IC MC1496P modulator/demodulator The reference wave is instead referenced to the signal transmission itself. Binary phase key shifting would use the reference phase carrier transmission which would systemically be encoded with binary. (8 stage binary would divide the transmissible wave into sections of 8(360/8 binary at 0 45 90 135 180 225 270 and 315) With binary indicating phase changes at known phase shifts if these phase shifts are known from the instance of emission, as binary is received back the microprocessor within the mouthpiece 12 will know precisely at which time that section of the wave was received back. This shall be known as Binary timing comparison, the scope of any phase modulation, phase shifting, or shifting of phase angle shall not limit signal processing scope or ability. All analog and or digital data collected from aforementioned measuring devices will be transmitted to an exemplary microcontroller (48 pin TQFP CY8C4125AXI-473) which in store can be directed towards proprietary cloud-based servers or in application diagnostics and analysis. The aforementioned shall not limit the scope of construction. If at anypoint in development the aforementioned UV hybrid aligners gain motility towards tooth surface reflective measurements can be calculated into T.O.F. sensors when deemed necessary. Initially the patient will remove aligners subsequent to analysis of the dentition.

### Chip Based Precise Ultrasonic Sensing

One solution to the issue that is micro measurement time of flight sensing and/or imaging is the utilization of a specialized chip based ultrasonic/optical sensor. The usage of a chip-based sensor, which utilizes both optical and mechanical resonances, provides exceptional sensitivity and accuracy when compared to the sole utilization of mechanical waves or electromagnetic waves. This approach uses a lithographically fabricated device suspended from a silicon chip with thin tethers. The utilization of these sensors in comparison to the utilization of previously existing sensors is a third order of magnitude increase in accuracy and near ideal acoustic impedance matching. A drawing of the suspended spoked silica disk array will be given along with a flowchart of information processes. The listed method can employ the utilization of fiber optics or any form. of optical approach necessary to detect and analyze the vibrations or signatures received from the chip-based silica tethered disk. Emission points utilized within this sensing method can be emitted from an array of ultrasonic emitters and or piezoelectric elements within the mouthpiece or on the chipset for the purpose of aligning teeth and or measuring Newton based forces within the body. To allow for chipset-based manufacturing the utilization of an integrated optical waveguide (Single mode exemplary) shall be used this will allow for light wavelength attenuations at a known point. The Photo detector (PD) will account for observations made as the reflection or reception of known attenuated light (emitted from Integrated optical waveguide) reflects off the micro-disk surface or around the peripheral area. To account for lack of acoustic area sensing the utilization of spoked silicon tethers can be used along with an increase in the number of micro-disks on the chipset allowing for a complete reflective image of the patient's teeth. An exemplary reception approach will utilize the dispersive coupling approach (reception is held horizontal in relative position of the silica tethered disk. This approach shall not limit the micro measuring capabilities and/or methods of development of any chip based ultrasonic/optical approach.

In reference to the utilization of chipset nano positioners the application shall store position data relative to previous adjustments. This will allow for the nano positioners to take into account previously collected data to determine position relative to tooth surface. The utilization of EER (Envelope Elimination and Restoration) will utilize the concepts of splitting the complex modulated ultrasonic wave signatures into amplitude and phase modulated waves which in turn will allow for signal processing and known wavelength attenuation. Again, This approach shall not limit the micro measuring capabilities and/or methods of development of any chip based ultrasonic/optical approach.

### Pressure Differentiation and Calculation to Achieve Remote Analysis

Another applicable method to achieve remote monitoring of the complete dentition is the use of an expandable pressure membrane (liquid or gas filled) and thin film pressure sensors/Force sensing resistor or board mounted pressure sensors. The said membrane would be sectioned off within its interior at known volumes. When those known volumes are filled to a point of deduced expansion their total volume as a standard can be subtracted from there partial volume to determine location of teeth. This shall not limit the scope of items to be utilized to accurately align the dentition. In this adaptation a thin silicon capacitive pressure sensor with monocrystalline silicon membranes. As the silicon membrane expands in the aforementioned mouthpiece 12 an electronic form of pressure analysis will show the pressure variant related expansion of the aforementioned silicon membrane showing the exact location of each tooth within the patient's dentition reliant upon the electrical feedback given by any of the aforementioned circuit workings describing the relativity of force to electrical signals/outputs. All alignment will be predicted and analyzed via the proprietary information surrounding physics of lingual to buccal axis movement and/or mesial or distal rotation (counterclockwise or clockwise).

### Computational Volumetric Analysis of Objects/Teeth with Irregular Shape to Determine Density Derived Mass

The listed method will coincide with similar methods of finding the geometric area of irregular shapes with no exact formula. For example, a method in two-dimensional geometric analysis relies on computerized analytics of a small two-dimensional square of known length and width; the program then analyzes the irregular shapes outline and computes the number of smaller known squares to precisely fill that irregular shape. Thus, determining volume by the length and width measurements of known squares multiplied by the number of squares that fit into the irregular shape. The premise relies on a 3-dimensional model of a patients tooth or entire dentition. Once uploaded via CAD/CAM a written program will outline the outer most layer of the tooth shape. Just as in the prior method the only absolute difference is that the shape of known geometry is 3 dimensional so that Length, Width, and height of the box is taken into account. Once analysis of total number of shapes in the third dimension is calculated it is then multiplied by the respective known (length, width, and height) variables to determine via computation the exact volume of a single tooth or plurality of teeth. Once volume is determined mass can then be derived from density and or a range of densities of specific teeth. The equation associated with such calculations is listed below. The theoretical assignment of a cube shall not limit the scope of possibilities of known dimensional variables that could be used to calculate irregular volume.

Mass=(Density)×(Volume)

### Dentition Pressure Calculations Derived Mathematically

Methodology surrounding calculations of pressure applied to individual teeth have been central in focus for many large orthodontic companies. The centrism surrounding all respective aligner companies existent today utilize treatment software which focuses primarily on the aesthetic of tooth positioning and the dentition these methods fail to observe or account for patient safety, accuracy, and efficiency. They achieve this in steps without taking into account the calculations necessary to increase predictability, patient safety, or information provided to the practicing orthodontist. The latest attempts to increase this predictability focus on deriving pressure calculations central to the pressures that aligners apply on teeth via sensing of Radio Frequencies relative to the position of Anode and Cathode receptors, or other sensing methods within the aligners themselves. This being said, the proprietary addition of Newtonian physics equations (F=MA, volumetric and area calculations) will allow for accurate prediction and analysis of pressures that teeth will undergo both prior and during in vivo aligner therapies without any utilization of sensor technology or live position detection. However, it may be deemed beneficial to utilize any proprietary measures to verify these predicted calculations in a live form. Pressure is mathematically defined as a measure of Force divided by the unit of area that Force is being applied to. Force would be calculated relative to tooth positioning over a known time variable as it is exemplified throughout this intellectual property. Area of tooth surface would be calculated via a direct relation of computational volumetric analysis, or within an individual tooth or the entirety of the represented dentitions CAD/CAM file. All prior analysis and prepared treatment steps relative to aligner therapies would be achieved within treatment planning software to ensure that each step or change in aligners will be accurate, efficient, and safe. A professional will upload a patients CAD/CAM dentition scan to a non-transitory computer storage medium, all calculated force and area variables necessary to calculate various tooth movements and the like will be stored in the same manner. The professional then can either observe the selected movements of each individual tooth or the entirety of the dentition and its respective force and pressure calculations at each step and edit if deemed necessary, or an algorithm can select the safest and most efficient steps relative to tooth movement over a given period of time utilizing these calculations of Force and relative pressure. Net pressure values would then be calculated computationally as they would be relative to the force in units of Newton applied to each individual tooth divided by the area of the lingual and/or buccal (Front and rear, dorsal or Frontal) views of the observed tooth. If deemed necessary to ensure accuracy in pressure calculations, or any relative calculations listed herein the addition of the area of the observed tooth's root surface can be utilized additionally to the area coefficient. The utilized equation of pressure is listed below.

Pressure(Pascals)=Force(N)/Area(mm²)

### Stepwise Description of Processes:

The collection of accurate analysis of a patient's dentition at an introductory appointment will be crucial for the in-silica analysis and prediction portion of this methodology. CAD/CAM scans by way of polyvinyl siloxane impressions, or via dentition imaging and mouth scanning. Once properly taken the patients CAD/CAM files will be uploaded to a computer storage and relay medium. Computational regard of Volumetric calculations and surface area of the tooth's facia both above the gingival line and below the gingival line will be calculated. The practitioner or computer program will then a selected time interval and cycle of which to project the intended movement of the dentition (every 2 weeks exemplary). Acceleration will then be calculated computationally by selecting the intended time variable of movement in seconds (1,209,600 seconds for a 14-day continuous wear period. It shall be noted that calculations will be reliant on the patient wearing the in vivo aligner therapy as intended (on a 24-hour basis). Thus, the proprietary methods of analyzing the intended dentition movement in a live in vivo to in silica relation will be beneficial for verification of patient wear, and predicted pressure and displacement relations. The intentional distance of the selected tooth/teeth/or dentitions movement will then be calculated respective to the time frame intended (1 mm in 31 days; 0.2 mm in a 14-day period.). Acceleration will then be calculated computationally relative to the selected intended time variable and the intended horizontal axis movement.

Mass will then be calculated computationally in the same light. Computational irregular volumetric calculation will find volume of analyzed tooth via CAD/CAM file or other respective means. Mass will then be calculated by multiplying the selected volume by a range of densities respective to the enamel and dentin layers associated with the observed tooth substrate, or the density relative to the entirety of the tooth in question. The computed means will then be determined via algorithm or suggested adjustment values in the presence of a practicing professional to then relate those calculations of Mass and acceleration to the amount of Force applied to the observed tooth or dentition. The projected force will then be divided by the coefficient of area associated with the observed tooth or the entirety of the observed and intended movement associated with the dentition to derive the amount of pressure that will be applied to the patient's dentition within that time period (14 days exemplary).

### Database Collection and Comparison of Projected, Observed and Calculated Measurements

It may be deemed beneficial to collect and observe not only the calculated intentional movement of the observed dentition, but also to collect the observed results of the intended movement. Thus, the adherence and relay of noted measurements of said values relative to pressure, displacement and observed movement can/will be collected and observed algorithmically and/or computationally by statistical distribution based not only on respective tooth type, but also respective to things such as rate of hyalinization, tooth density, mass, and the ratio to pressure and successful movement as it would fall within a range relative to another patient's experience.

### Data Relay and Comparative Analysis

For the purpose of mass data distribution, a selected or developed web or server based encrypted API can be utilized to transmit data collected from any complex data derived form listed within this disclosure (Mouthpieces) the purpose of this is to create an independent trafficking route for complex forms of this listed data which can be encrypted at the patient's application, which is connected to the Web based data transmissive API. If necessary, the utilization of a small Raspberry Pi or similar method of computation can be utilized. For the categorization of said data environments listed sensors inside proprietary mouthpieces can be numbered concurrent with the standard numerical method of numbering teeth in dentistry. For example, teeth 6, 11, 22, and 27, which are all anterior canines, also exemplifies sensor numbers 6, 11, 22, and 27.

Another possible benefit could be to directly compare data of like teeth on the same or opposing arches. this could easily be achieved by comparing alignment/displacement data from tooth/sensor 6 and tooth/sensor 11, or 22 and 27. This is exemplary the same could be done by classifying the different types of the teeth against respective types of symmetry (upper right 2nd molar compared to upper left 2nd molar and/or lower left and lower right 2nd molars)

### Fourier Transformations

If deemed beneficial for the interpretation of any listed waveforms within this disclosure the utilization of Fourier transformations and/or concurrent Fourier series can be utilized to further interpret the reception and/or emission of any listed wave form. A Fourier transformation is utilized primarily in NMR spectroscopy of organic chemistry molecules for classification in specific frequencies. It also is used to identify specific pitches of identifiable intensity in musical chords (categorize and identify). It simply changes an emitted/received wave from a time domain to a frequency domain and back. For the utilization of signal processing, it could be utilized to transform a frequency in a known wave from a time domain and back meaning it could easily be identified in the utilization of TOF sensors by simply keying into a portion of a wave at its highest pitch or intensity. This can be done by simply deconstructing any emitted waveform into a series of a sum of many sine waves if an emitted wave is then interpreted as the longest wavelength of one of those deconstructed sine waves signal processing for the purposes of Time of Flight will benefit directly.

### Time Sequencing

Time sequencing may be utilized as a signal processing method in any respect deemed beneficial and relative to this intellectual property. Time sequencing and the like are used and relied upon heavily within the biomedical engineering industry and are used as a stronghold in EKG and ECG signal processing (heart monitors). The most prevalent representation of the benefit to be gained has been found within its utilization in sound based (ultrasonic) emission and concurrent Time of Flight reception. Time series; a sequence of data indexed by successive time points can be utilized within sources sampled over a short time. For utilization within sound-based time of flight sensors an exemplary sound-based emission source would activate directed towards the surface of a patient's corresponding tooth. A multiplicity of emitted and received data points with listed magnitudes, frequency, and of set waves would be collected. Once collected the utilization of a lowpass filter (attenuates higher undesired frequencies) and/or the utilization of a determined high-pass filter (attenuates lower undesired frequencies) can be utilized to intentionally increase the stopband above or below undesired frequency thresholds. Filters can be adjusted and designed accordingly to the best suited frequency or magnitude that would be observed. Desired and raw signal data relative to each TOF sensor and its corresponding emission and reception data can be stored in a non-transitory computer storage medium, cloud-based server, or web-based API. The selected and most prime combination of low-pass and high-pass filtering can then be applied via computer algorithm, program integrated into web-based API, or cloud-based server. This is an exemplary approach to signal processing and is not intended to limit the scope of future methodology in signal processing deemed beneficial.

### Utilization of Wave Properties in Time-of-Flight Sensing

It is of benefit to alter the methodology of which Time of Flight sensors calculate distance between sensor emission point, and tooth reflectance surface to calculate distance therein. One such alteration is to format the respective ultrasonic MEMS sensors to detect the amplitude, period, cycle, frequency or any respective wave calculation of received wave patterns individually, or as a collected average of like data points post Low pass and High pass filtering (to eliminate extraneous variables from calculations). Amplitude data capture is beneficial from the perspective of filtering to allow for more precise mathematical determinations of a sensor's respective emission and reception data points. Amplitudes of higher or lower variables would be excluded in calculations as extraneous variables. The equation which allows for mathematical accuracy and precision in calculation of wavelength is V=Lambda(F) (V=Velocity Lambda=Wavelength F=Frequency). Velocity would be referenced relative to the speed of sound within the mouth which has already been listed and calculated precisely at various temperature points. Frequency would be known relative to the ultrasonic sensor being utilized. Wavelength (lambda) could then be mathematically calculated to determine with mathematical precision the length of a single wave respective of frequency and speed of sound. The known time variable in (milliseconds/nanoseconds/picoseconds) from the emission point and reception point divided by two would allow for an accurate analysis of the time taken for observation relative of emission to tooth surface. Period, or the time taken for one cycle of a known wave to complete would be calculated as 1/Frequency. The distance that emitted sound would travel during one period is defined as the wavelength. The given Frequency of an observed sound-based sensor would display the number of cycles a wave would undergo in one second. Once period (time taken to complete one cycle) is calculated it is then multiplied by the time variable (time from tooth surface to sensor) to determine the exact number of cycles that a signal would undergo during an observed time period. Distance would then be the product of the exact number of cycles in an observed time period and the wavelength of those cycles. Thus, allowing a mathematical calculation derived from wavelength and wave cycles over a given distance. This example is not to limit the scope of mathematical sinusoidal calculation as it is beneficial to accurate distance calculations. All counterparts observed, but not mentioned relative to a sound wave (period, Crest, Trough, wavelength, Frequency, wave angle, wave velocity etc.) may be utilized in part for the benefit of signal processing and calculation.

### Utilization in a Non-Remote Form

All the previous/derived methods listed within can be constructed in methodology to be used within any office setting via a large-scale device. All the previous/derived methods can be utilized within current/future X-ray machines and/or 3D scanners or any large scale in office device under direct licensure of this intellectual property. In short, if licensure is granted utilization of any of these principles, theories, or the like can be utilized in the construction of devices used in a non-remote form to analyze tooth movement and force vectors within a physical building. The data flows will remain the same, however the data will be stored either within an encrypted server on site/off site to be accessed by a certified dental professional.

All listed forms of analysis where unspecified can be utilized to track and predict movement/di splacement/hyalinization/de-hyalinization/or any methodology of predictive treatment analysis of the dentition regardless of how that movement occurs (Brackets. wires, headgear, CuNiTi, lingual appliance, aligner, or the like).

Although several preferred embodiments have been described in detail, it should be understood that various changes, substitutions, and alterations can be made therein without departing from the spirit and scope of the invention as defined by the appended claims.

What is claimed is:

1. A method of moving at least one tooth within a plurality of teeth, comprising:
determining the mass of the tooth;
determining the initial position of the tooth;
calculating a first force necessary to move the tooth a first distance in a first period of time using Newtonian physics equations; and
applying the first force to the tooth for the first period of time by using an aligner tray.

2. The method of claim 1 further comprising:
determining a second position of the tooth after expiration of the first period of time;
calibrating the calculation of force by adjusting the Newtonian equations to compensate for the actual position of the tooth as compared to the predicted position of the tooth;
calculating a second force needed to move the tooth a second distance in a second period of time using the calibrated Newtonian equations; and
applying the second force to the tooth for the second period of time using an aligner tray.

3. The method of claim 1 further comprising providing an aligner tray having a selected one of retro-reflective pigments, pressure sensitive polymers or holographic films.

4. The method of claim 1 wherein the initial position of the tooth is determined by placing the plurality of teeth into a mouthpiece having an emission source and a receiving sensor mounted inside the mouthpiece.

5. The method of claim 4 wherein the mouthpiece connects wirelessly to computing device.

6. The method of claim 5 wherein the mouthpiece has a wired connection to a computing device.

7. A device for determining the position of a tooth within a plurality of teeth, comprising:
a U-shaped mouthpiece body having a channel within which to receive the teeth;
one or more emission sources mounted to the inside of the mouthpiece body to direct waves into the channel;
one or more receiving sensors mounted to the inside of the mouthpiece body for receiving reflected emissions;
memory for storing the reflected emission data; and
a processor for calculating and determining the position of the tooth.

8. The device of claim 7 wherein the emission source is a light emitting source.

9. The device of claim 8 wherein the light emitting source is a diode.

10. The device of claim 8 wherein the light emitting source and the receiving sensor are time-of-flight sensors.

11. The device of claim 7 further comprising an aligner tray positioned on the teeth having a selected one of retro-reflective pigments, pressure sensitive polymers or holographic films.

12. The device of claim 7 further wherein the mouthpiece is operatively connected to a computing device which houses the memory and processor.

13. The device of claim 12 wherein the computer determines the position of the tooth by from the reflected emission data.

14. A system for moving one or more teeth within a plurality of teeth, comprising:
a mouthpiece, which comprises:
a U-shaped mouthpiece body having a channel within which to receive the teeth;
one or more light sources for emitting light within the channel; and
one or more indentation mechanisms capable of extending into the channel; and
an aligner tray within the U-shaped channel of the mouthpiece comprised of a selected one of a polymer or silicone;
wherein the light from the light source emits a first wavelength of light to soften the aligner tray, then the indentation mechanism applies pressure to the aligner tray to reshape the aligner tray, and then the light source emits a second wavelength of light to harden the aligner tray.

15. The system of claim 14 wherein the light source is a diode.

16. The system of claim 15 wherein the light source and indention mechanism can be controlled remotely.
